(19) United States
(12) Patent Application Publication
(10) Pub. No.: US 2009/0196877 A1
(43) Pub. Date: Aug. 6, 2009

(54) Novel Method to Increase Memory T Lymphocytes and Enhance Their Functions

(75) Inventor: Lieping Chen, Sparks Glencoe, MD (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 12/085,969

(22) PCT Filed: Dec. 4, 2006
(86) PCT No.: PCT/US06/46279

§ 371 Date: Feb. 17, 2009

Related U.S. Application Data
(60) Provisional application No. 60/741,816, filed on Dec. 2, 2005.

(51) Int. Cl. A61K 39/395; C12Q 1/68; A01K 67/027; C07K 16/18

(52) U.S. Cl. 424/158.1; 435/6; 800/3; 530/387.1

(57) ABSTRACT

The invention generally features compositions and methods that are useful for increasing immune function. Such methods can be employed to enhance innate immunity for the prevention or treatment of pathogen infections (e.g., bacterial, viral, or fungal infections), lymphopenia, or cancer by stimulating a CD1 37 polypeptide expressed on an immune cell, such as a memory T cell. The invention further comprises a mouse lacking detectable levels of CD 137, cells derived from the mouse, and methods of producing additional knockouts animals.

### CROSS-REFERENCE TO RELATED APPLICATION

This application claims the benefit of the following U.S. Provisional Application No. 60/741,816, the entire disclosure of which is hereby incorporated in its entirety.

### STATEMENT OF RIGHTS TO INVENTIONS MADE UNDER FEDERALLY SPONSORED RESEARCH

This work was supported by the following grants from the National Institutes of Health, Grant No. CA85721. The government may have certain rights in the invention.

### BACKGROUND OF THE INVENTION

Memory T cells respond to repeated antigen assaults by generating effector T cells. Memory T cells, therefore, represent important host defense mechanisms of adaptive immunity against infection and malignancies. In normal individuals memory T cells are present in greater numbers than natural killer cells. Thus, the antigen-independent innate immunity function of memory T cells represents a powerful host defense mechanism that can be used to combat pathogen infections, including bacterial and viral infections. Innate immunity is also important in combating neoplasia. Methods of inducing or enhancing an innate immune response in a subject are required.

### SUMMARY OF THE INVENTION

As described below, the present invention features methods for enhancing immune function. In particular, the invention provides prophylactic and therapeutic methods that enhance resistance to diseases, such as pathogen infections and neoplasia.

In one aspect, the invention generally features a method of increasing innate immune function in a subject identified as in need thereof, the method comprising contacting a memory T cell of the subject with an agent that specifically binds CD137; and inducing memory T cell proliferation in the subject, thereby increasing innate immunity. In one embodiment, the method prevents the onset of neoplasia, lymphophenia, or pathogen infection in a subject at risk thereof.

In another aspect, the invention generally features a method of increasing memory T cell proliferation, the method comprising contacting a memory T cell expressing CD137 with an agent that activates CD137; and inducing memory T cell proliferation. In one embodiment, the method prevents the onset of neoplasia, lymphophenia, or pathogen infection in a subject at risk thereof.

In yet another aspect, the invention features a method of treating or preventing a pathogen infection in a subject in need thereof, the method comprising: administering to the subject an agent that specifically binds CD137 on a memory T cell; and inducing an innate immune response in the subject, thereby treating or preventing a pathogen infection. In one embodiment, the pathogen infection is bacterial (e.g., any one or more of Aerobacter, Aeromonas, Acinetobacter, Actinomyces israelli, Agrobacterium, Bacillus, Bacillus antracis, Bacteroides, Bartonella, Bordetella, Bortella, Borrelia, Brucella, Burkholderia, Calymmatobacterium, Campylobacter, Citrobacter, Clostridium, Clostridium perfringers, Clostridium tetani, Cornyebacterium, corynebacterium diphtheriae, corynebacterium sp., Enterobacter, Enterobacter aerogenes, Enterococcus, Erysipelothrix rhusiopathiae, Escherichia, Francisella, Fusobacterium nucleatum, Gardnerella, Haemophilus, Hafnia, Helicobacter, Klebsiella, Klebsiella pneumoniae, Lactobacillus, Legionella, Leptospira, Listeria (e.g., Listeria monocytogenes), Morganella, Moraxella, Mycobacterium, Neisseria, Pasteurella, Pasturella multocida, Proteus, Providencia, Pseudomonas, Rickettsia, Salmonella, Serratia, Shigella, Staphylococcus, Stentorophomonas, Streptococcus, Streptobacillus moniliformis, Treponema, Treponema pallidium, Treponema pertenue, Xanthomonas, Vibrio, and Yersinia. In one embodiment, the bacterial infection is a Listeria monocytogenes infection. In another embodiment, the viral infection is an infection with any one or more of Retroviridae, HIV-1, Picornaviridae, Calciviridae, Flaviridae, Coronoviridae, Filoviridae, Paramyxoviridae, Orthomyxoviridae, Bungaviridae, Arena viridae, Birnaviridae; Hepadnaviridae, Parvovirida, Papovaviridae, Adenoviridae, Herpesviridae, Poxyiridae and Iridoviridae. In one embodiment, the viral infection is a Human immunodeficiency virus infection or HIV/AIDS). In another embodiment, the pathogen infection is a fungal infection. In one embodiment, the method prevents the onset of pathogen infection in a subject at risk thereof.

In yet another aspect, the invention features a method of treating or preventing a neoplasia in a subject in need thereof, the method involving administering to the subject an agent that specifically binds CD137 on a memory T cell; and inducing an innate immune response in the subject, thereby treating or preventing a neoplasia (e.g., a lymphoma) In one embodiment, the method prevents the onset of neoplasia in a subject at risk thereof.

In yet another aspect, the invention features a method for increasing homeostatic proliferation in a subject identified as in need thereof, the method comprising contacting a memory T cell expressing CD137 with an agent that activates CD137; and inducing memory T cell proliferation. In one embodiment, the method prevents the onset of in a subject at risk thereof.

In various embodiments of the previous aspects, the agent is a monoclonal antibody, CD137 ligand, or mimetic thereof that specifically binds to CD137 and acts as an agonist thereof.

In yet another aspect, the invention features a method for identifying an agent that modulates innate immunity, the method comprising the steps of providing a cell expressing a CD137 nucleic acid molecule; contacting the cell with a candidate compound; and comparing CD137 nucleic acid molecule expression in the contacted cell with a reference level of expression, wherein an alteration in CD137 nucleic acid molecule expression identifies the candidate compound as a candidate compound that modulates innate immunity. In one embodiment, the method identifies an agent that increases or decreases CD137 transcription. In yet another embodiment, the method identifies an agent that increases or decreases translation of an mRNA transcribed from the CD137 nucleic acid molecule. In other embodiments, the method identifies an agent (e.g., monoclonal antibody, CD137 ligand, or mimetic thereof) that specifically binds to CD137 and induces memory T cell proliferation.

In yet another embodiment, invention features a method for identifying an agent that modulates innate immunity, the method comprising the steps of providing a cell expressing a CD137 polypeptide; contacting the cell with a candidate compound; and detecting an alteration in the level of CD137 polypeptide in the cell contacted with the candidate compound relative to a reference level, wherein an alteration in the level of CD137 polypeptide identifies an agent that modulates innate immunity.

In another aspect, the invention features a method for identifying an agent that modulates innate immunity, the method comprising the steps of providing a cell expressing a CD137 polypeptide; contacting the cell with an agent; and comparing the biological activity of CD137 polypeptide in the cell contacted with the candidate compound with the biological activity in a control cell, wherein an increase in the biological activity of the host response to pathogen identifies the candidate compound as a candidate compound that modulates innate immunity.

In yet another aspect, the invention provides a method for identifying an agent that increases a host response to a pathogen, the method comprising the steps of providing a cell expressing a CD137 polypeptide; contacting the cell with a candidate compound; and detecting binding of the CD137 polypeptide with the candidate compound, wherein a compound that binds a CD137 polypeptide is useful for increasing a host response to a pathogen. In one embodiment, the agent is a polynucleotide, polypeptide, or small compound. In another embodiment, the method further involves the step of contacting the agent with a memory T cell and assaying cell proliferation. In yet another embodiment, the method is carried out in vivo or in vitro.

In various embodiments of the previous aspects, the identified agent is useful as a prophylactic that prevents a neoplasia, lymphopenia, or pathogen infection in a subject at risk thereof.

In yet another aspect, the invention features a mouse containing a mutation in a gene that encodes a murine CD137/4-1BB polypeptide. In one embodiment, the mouse fails to express detectable levels of the polypeptide. In another embodiment, the mouse is generated by inducing homologous recombination in an embryonic stem cell. In yet another embodiment, the mouse contains a neo-resistance cassette in exons 1-6 of endogenous CD137. In yet another embodiment, the mouse is a knockout mouse.

In a related aspect, the invention provides a cell or cell line isolated from the mouse of the previous aspect.

In yet another aspect, the invention features a method of screening for a compound that modulates an immune response, the method comprising, exposing the mouse of the previous aspect, or a cell derived therefrom, to a compound, and determining the level of immune response in the mouse, wherein an increase in the immune response as compared to an untreated mouse indicates that the compound enhances an immune response.

In another related aspect, the invention features a method of producing the mouse of the previous aspect, the method involving generating a targeting plasmid comprising a CD137 gene comprising a mutation; contacting an embryonic stem cell of a wild type mouse with the targeting plasmid; injecting the targeted embryonic stem cell into a blastocyst of a host mouse to produce a zygote; transplanting the zygote into a host mouse; obtaining a founder mouse carrying the knockout; and breeding the founder mouse to obtain a mouse that lacks detectable levels of CD137.

In another aspect, the invention features an isolated antibody that specifically binds human CD137. In one embodiment, the antibody is a monoclonal antibody that acts as a CD137 agonist.

In various embodiments of any of the above aspects, the method prevents a disease or disorder. In still other embodiments, the method increases the proliferation of CD44^{hi} cell (e.g., a memory T cell), or increases cytokine secretion or cytolytic activity for tumor cells. In still other embodiments, the induction of the innate immune response, T cell proliferation, cytokine secretion, or cytolytic activity occurs in the absence of T cell receptor, T cell response, or T cell receptor signalling. One measure of T cell receptor signalling is CD69 and CD25 upregulation. In other embodiments of the above-aspects, the memory T cell is in vitro or in vivo. In still other embodiments of the above-aspects, the method further contains delivering the memory T cell to a subject identified as in need of an increase in innate immunity. In still other embodiments of the above-aspects, the agent is an antibody that specifically binds human CD137, such as a human CD137 monoclonal antibody that acts as a CD137 agonist. In one embodiment, the memory T cell division occurs in a self major histocompatibility cell (MHC) independent process. In other embodiments of the above-aspects, the method increases the number of CD137 positive CD44^{hi} cells. In still other embodiments of the above-aspects, the method increases memory T cell number by at least 2-fold. In still other embodiments of the above-aspects, the induction of an innate immune response results in an amelioration of the pathogen infection (e.g., Listeria monocytogenes) or neoplasia (e.g., lymphoma). In still other embodiments of the above-aspects, the method reduces the number of pathogens or the rate of pathogen proliferation. In still other embodiments of the above-aspects, the method reduces the rate of neoplastic cell proliferation or reduces the size of the neoplasia. In other embodiments of the above-aspects, the subject is undergoing chemotherapy, is diagnosed with a or has a chronic infection.

### DEFINITIONS

By “memory T cell” is meant a T cell capable of an anamnestic proliferative response to an antigen in vitro.

By “CD137 polypeptide” is meant a polypeptide or fragment thereof having at least 85% identity to the CD137 polypeptide

By “CD137 monoclonal antibody 2A” is meant the antibody described by Wilcox et al., J. Clin. Invest. 109, 651-659 (2002a) and by Sun et al., Nat. Med. 2002 December; 8 (12):1405-13 that specifically binds CD137. By “innate immunity” is meant a native or natural immunity whose defense mechanisms are present prior to exposure to infectious microbes or foreign substances.

By “CD44^{hi}”, is meant a T cell that expresses an increased level of CD44 as evaluated by flow cytometric analysis, CD44, is variably expressed on T cells, and flow cytometric analysis is used to define two separate CD4+ subsets: CD44^{lo} and CD44^{hi}. The activation of naive cells via the T-cell receptor induces the increased expression of CD44, with subsequent conversion from the naive (CD44^{lo}) to the activated (CD44^{hi}) phenotype.

By “CD137 agonist” is meant an agent that specifically binds to CD137 and causes an increase in memory T cell proliferation.

By “identified as in need of an increase in innate immunity” is meant that a physician or other clinician has selected the subject as likely to benefit from a prophylactic or therapeutic that enhance the subject's innate immune response. Criterion for selection include, but are not limited to, subject's identified as having or at risk of developing a pathogen infection, including a chronic infection, having or at risk of developing a neoplasia, undergoing chemotherapy, having or at risk of developing lymphopenia. Other patients that might benefit from therapeutic methods described herein include those having a reduced number of memory T cells (or their progenitor cells) or a reduced efficacy of immune response.

By “homeostatic proliferation” is meant T-cell proliferation under conditions of lymphopenia.

By “lymphopenia” is meant the presence of a reduced number of lymphocytes in the circulating blood of a subject relative to the number present in a normal control subject. Lymphopenia can be caused by various types of chemotherapy, such as with cytotoxic agents or immunosuppresive drugs. Some malignancies in the bone marrow will also cause lymphopenia. A decreased number of lymphocytes (notably T cells) is present in those with AIDS. Subjects exposed to radiation may also exhibit lymphopenia. Lymphopenia may be present as part of a pancytopenia, where the total number of blood cells is reduced. This can occur in marrow failure.

By “ameliorate” is meant decrease, suppress, attenuate, diminish, arrest, or stabilize the development or progression of a disease.

By “agent” is meant any small molecule chemical compound, antibody, nucleic acid molecule, or polypeptide, or fragments thereof.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens. If desired, antibodies of the invention are conjugated to a detectable label.

A “detectable level” as used herein, means a level of polypeptide or polynucleotide that is detectable by standard techniques currently known in the art or those that become standard at some future time, and include for example, Western blot, ELISA, SDS-PAGE, radioimmunoassay, differential display, RT (reverse transcriptase)-coupled polymerase chain reaction (PCR), Northern Blot, or any other method known in the art. The degree of differences in expression levels need only be large enough to be visualized or measured via standard characterization techniques.

By “disease” is meant any condition or disorder that damages or interferes with the normal function of a cell, tissue, or organ. Examples of diseases include bacterial invasion or colonization of a host cell.

The term “expression” refers to the biosynthesis of a gene product. For example, in the case of a structural gene, expression involves transcription of the structural gene into mRNA or the translation of mRNA into one or more polypeptides.

By “an effective amount” is meant the amount of a required to ameliorate the symptoms of a disease relative to an untreated patient. The effective amount of active compound(s) used to practice the present invention for therapeutic treatment of a neoplasia, lymphopenia, or pathogen infection varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 700%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “increase” is meant any positive alteration in a parameter. An increase may be by 10%, 25%, 50%, 75%, or even by 100% or more relative to a reference level.

By “increasing memory T cell proliferation” is meant increasing the rate of cell proliferation or increasing the absolute number of memory T cells present in a subject relative to an untreated control subject.

By “isolated nucleic acid molecule” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By “marker” is meant any protein or polynucleotide having an alteration in expression level or activity that is associated with a disease or disorder.

By “mutation” is meant any change in an amino acid or nucleic acid sequence. Exemplary mutations include insertions, deletions, frameshift mutations, or missense mutations.

By “neoplasia” is meant a disease that is caused by or results in inappropriately high levels of cell division, inappropriately low levels of apoptosis, or both. For example, cancer is an example of a neoplasia. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

By “pathogen” is meant any bacteria, viruses, fungi, or protozoans capable of interfering with the normal function of a cell. Exemplary bacterial pathogens include, but are not limited to, Aerobacter, Aeromonas, Acinetobacter, Actinomyces israelli, Agrobacterium, Bacillus, Bacillus antracis, Bacteroides, Bartonella, Bordetella, Bortella, Borrelia, Brucella, Burkholderia, Calymmatobacterium, Campylobacter, Citrobacter, Clostridium, Clostridium perfringers, Clostridium tetani, Cornyebacterium, corynebacterium diphtheriae, corynebacterium sp., Enterobacter, Enterobacter aerogenes, Enterococcus, Erysipelothrix rhusiopathiae, Escherichia, Francisella, Fusobacterium nucleatum, Gardnerella, Haemophilus, Hafnia, Helicobacter, Klebsiella, Klebsiella pneumoniae, Lactobacillus, Legionella, Leptospira, Listeria (e.g., Listeria monocytogenes), Morganella, Moraxella, Mycobacterium, Neisseria, Pasteurella, Pasturella multocida, Proteus, Providencia, Pseudomonas, Rickettsia, Salmonella, Serratia, Shigella, Staphylococcus, Stentorophomonas, Streptococcus, Streptobacillus moniliformis, Treponema, Treponema pallidium, Treponema pertenue, Xanthomonas, Vibrio, and Yersinia.

Examples of viruses that have been found in humans include but are not limited to: Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By an “isolated polypeptide” is meant a polypeptide of the invention that has been separated from components that naturally accompany it. Typically, the polypeptide, such as an antibody, is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “subject” is meant a mammal, including, but not limited to, a human or non-human mammal, such as a bovine, equine, canine, ovine, or feline.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

A “targeting vector” is a nucleic acid molecule, for example, a plasmid that includes a sequence capable of recombining with a target sequence. Targeting vectors typically contain (i) one or a small number of restriction endonuclease recognition sites at which foreign DNA sequences can be inserted in a determinable fashion without loss of an essential biological function of the vector, and (ii) a marker gene that is suitable for use in the identification and selection of cells transformed or transfected with the targeting vector. Marker genes include genes that provide neomycin, tetracycline, or ampicillin resistance, for example.

“Therapeutic agent” means a substance that has the potential of affecting the function of an organism. Such an agent may be, for example, a naturally occurring, semi-synthetic, or synthetic compound. For example, the candidate agent may be a drug that targets a specific function of an organism. A test agent may also be an antibiotic or a nutrient A therapeutic agent may decrease, suppress, attenuate, diminish, arrest, or stabilize the development or progression of disease, disorder, or infection in a eukaryotic host organism.

By “reference” is meant a standard or control condition.

By “isolated nucleic acid molecule” is meant a polynucleotide that is isolated from the flanking genomic regions that normally accompany it. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. C. in 750 mM NaCl, 75 mM trisodium citrate, and 1% SDS. In a more preferred embodiment, hybridization will occur at 37° C. C. in 500 mM NaCl, 50 mM trisodium citrate, 1% SDS, 35% formamide, and 100. mu.g/ml denatured salmon sperm DNA (ssDNA). In a most preferred embodiment, hybridization will occur at 42° C. C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 M NaCl, 3 mM trisodium citrate, and 0.1% SDS. In a more preferred embodiment, wash steps will occur at 42.degree. C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

### BRIEF DESCRIPTION OF THE DRAWINGS

FIGS. 1a and 1b show the nucleic acid and amino acid sequences of mouse T-cell receptor 4-1BB protein mRNA (NCBI Reference Nos. J04492; P20334) and human CD137 (NCBI Reference Nos. NM_{—}001561 and NP_{—}001552, respectively).

FIGS. 2a-2f show that the CD137 agonistic monoclonal antibody (2A mAb) selectively stimulates proliferation of memory T cells. C57B16 (B6) (FIGS. 2a-2e) or C3H/HeJ (FIG. 2f) mice were injected with 100 μg of control rat IgG or 2A mAb on day 0 and day 2 post-infection, and fed with drinking water with 0.8 mg/ml BrdU from day 3 to day 7. Splenocytes (FIGS. 2a, b, f) or intrahepatic lymphocytes (FIGS. 2c, d, e) were harvested on day 7 and stained for CD8, CD4, CD44 and BrdU by flow cytometry. FIG. 2a includes four panels showing the results of flow cytometry analysis. Data were presented by gating on CD8 or CD4. FIG. 2b includes four graphs showing that the percentages of CD44^{hi} or CD122^{hi} cells present in CD8+ or CD4+ T cell subsets derived from spleens (FIG. 2b) or livers (FIG. 2d) increase significantly from days 5 through 8, after 2A mAb. The number of total intrahepatic lymphocytes as well as CD4+ and CD8+ subsets on day 7 is also shown in FIG. 2e. The results shown are from one representative experiment. Three independent experiments with three or five mice each were carried out and similar results were obtained. *, p<0.05, **, p<0.001.

FIG. 3 includes three graphs that show accumulation of memory T cells in the spleens upon CD137 monoclonal antibody injection (2A). Naive B6 mice were injected intraperitoneally (i.p.) with 0.1 mg of control rat IgG or 2A monoclonal antibody (mAb) on day 0 and day 2. The numbers of CD44^{hi} or CD122^{hi} cells in CD8+ or CD4+ T cell subsets in spleens was counted on day 5 and day 8. The data shown are the average of five mice in each group.

FIG. 4 shows that CD137 mAb (2A mAb) treatment does not induce the expression of the T cell activation markers CD69 and CD25. Naïve B6 mice were treated i.p. with 0.1 mg/mouse 2A mAb or Rat Ig control mAb on day 0 and day 2. On day 5, spleen cells were harvested and stained for CD8, CD25 or CD69 using specific mAb. FIG. 4 shows a panel of four graphs where the data is shown by selecting CD8+ cells.

FIG. 5 shows that CD137 stimulation induces proliferation of memory but not naïve T-cells. B6 mice containing naïve (upper panels) or memory OT-1 x RAG-1 KO TCR transgenic T cells (lower panels) were treated with control mAb (Rat Ig) or 2A mAb. The mice were fed with BrdU-containing drinking water for 5 days. Spleen cells were harvested and stained for CD8, OT-1 tetramer and anti-BrdU. FIG. 5 shows four panels where data was gated on CD8+ and tetramer+ cells. Results shown are one representative of two independent experiments with three mice each. *, p<0.05, memory cells treated with CD137 mAb versus control antibody.

FIGS. 6 a-6f shows generation and characterization of CD137 KO mice and the effect of CD137 agonistic mAb. FIG. 6a shows the targeting map of the CD137 genomic locus. The signal peptide with the ATG starting code and first 6 exon encoding extracellular and transmembrane regions of murine CD137 were replaced with a Neo cassette. A short open bar labeled as “Probe” indicates the position of 3′ end probe for screening of ES cells, and “PCR” indicates the position of PCR products in screening of CD137 deficient mice using primers. Shaded boxes represent exons within murine CD137 open reading forme. FIG. 6b shows Southern blotting of heterozygous and homozygous CD137 mutants in the genomic DNA from targeted embryo stem (ES) cells. The upper band (6691 bp) shows the targeted fragment and the lower one (6147 bp) represents the one from normal genome. FIG. 6c is a panel of four graphs. Splenocytes from wild type (WT) B6 or CD137 KO mice were activated by ConA for 24 hours and live cells were stained for CD137 or PD-1 gated on CD3+ cells by specific mAb (open area) or control antibodies (filled area), and subsequently analyzed by flow cytometry. FIG. 6d shows two graphs. Total T cells were purified from lymph nodes of CD137KO or WT control mice and were activated by Con A or plate-bound CD3 mAb at indicated concentrations. [³]TdR thymidine was included in the cultures 16 hours before harvesting. The results are from one representative of two independent experiments with similar results. FIG. 6e shows four panels. Splenocytes from untreated WT or CD137 KO mice were stained for CD44 and CD62 ligand (CD62L) that were gated on CD8+ or CD4+ cells respectively. The results are from one representative of two independent experiments with three mice each. FIG. 6f consists of four panels that show that in the absence of CD137 on T cells, the anti-CD137 mAb is without effect. CD8+ T cells from WT or CD137KO mice (Thy1.2+) were transferred into B6/Thy1.1 congenic mice and subsequently treated with rat IgG or 2A mAb as described before. Spleen cells were harvested, counted and stained for CD8, Thy1.2 and CD44. The expression of CD44 and Thy1.2 in gated CD8+ cells is shown. The numbers are presented as a percentage of each subset in CD8+ cells. The numbers within parentheses represent absolute numbers of cells in the whole spleen. The results are from one representative of two independent experiments with three mice each. *, p<0.05.

FIG. 7 shows that memory T cells in CD137 KO mice respond normally to polyinosinic:polycytidylic acid (poly I:C), which is a synthetic double-stranded RNA that is used experimentally to model viral infections in vivo, but not to 2A mAb. CD137 KO mice or wild type (WT) mice were injected i.p. with control mAb (Rat Ig), poly I:C or 2A CD137 mAb on day 0 and fed with BrdU (full name: Bromodeoxyuridine) from day 1 to day 4. Spleen cells were harvested on day 4 and stained for CD8, CD44 and BrdU. The % of CD44+ cells was labeled. The data represents] gating of CD8+ cells, and is representative of two independent experiments.

FIG. 8 provides four panels showing the effects of 2A CD137 mAb on naïve T cell homeostasis in lymphopenic mice. 1×10⁶ Carboxy-Fluorescein diacetate, Succinimidyl Ester-labeled naïve OT-1 x RAG1 KO T cells were adoptively transferred into sublethally-irradiated B6 mice. 100 μg rat IgG or 2A mAb was injected interperitoneally on day 0 (upper panels) or day 7 (lower panels) after cell transfer. Spleen cells were prepared at day 6 after treatment and analyzed by flow cytometry. Histogram plots of CFSE intensity of transferred OT-1 cells (gated on CD8+ OT-1 tetramer+) in spleen is shown. The results are one representative of three independent experiments with similar results. **, p<0.001.

FIGS. 9a-9c shows CD137 stimulation-induced proliferation of memory OT-1 T cells is independent on MHC, IL-15 and IFN-γ. FIG. 9a is a panel of two graphs. CFSE-labeled memory OT-1 cells were adoptively transferred into H-2 Kb KO mice and subsequently treated with CD137 mAb or control antibody on day 1 and day 3. On day 7, spleen cells were harvested and stained for CD8, OT-1 tetramer. CFSE intensity of transferred memory OT-1 cell was shown by FACS, gated on CD8 and OT-1 tetramer+ cells. The results are from one representative of two independent experiments with three mice each group. *, p<0.05. FIG. 9b shows two panels. B6 mice containing memory OT-1 cells were injected with anti-H-2Kb blocking mAb on day −1 and 2. On day 0 and day 2, mice were treated with CD137 mAb or control mAb respectively and fed with PBS containing BrdU as shown previously. Spleen cells were prepared at day 7 after treatment and analyzed by flow cytometry. Histogram plots of CFSE intensity of transferred OT-1 cells (gated on CD8+ OT-1 tetramer+) in the spleen is shown. The results shown are from one representative of two independent experiments with three mice each group. *, p<0.05. FIG. 9c is a graph. IL-15 KO and IFN-γ KO mice were treated with indicated mAb and fed with BrdU as shown on FIG. 1. Data shown represent % of BrdU+ cells gated on CD8+ CD44^{hi} portion. The results are from one representative experiment of two independent experiments carried out using three mice for each experiment. *, p>0.05, no significant difference among each groups. **, p<0.001, CD137 mAb versus control mAb.

FIG. 10 shows that CD40 ligand (CD40L) is not required for CD137 mAb-stimulated memory T cell proliferation. Naïve B6 mice were treated i.p. with 0.2 mg/mouse of control (None) or MR1 (anti-CD40L neutralizing mAb) on day 1 and day 3. On day 0, the mice were treated i.p. with 2A mAb or control mAb (Rat Ig) at 0.1 mg/mouse and subsequently fed with BrdU as indicated previously in FIG. 1a On day 7 spleen cells were harvested and stained for CD8, CD44 and BrdU. Data shown represents cells gated on CD8+ cells and are a representative of three mice in each group.

FIG. 11a-11f shows that CD137 mAb confers on naïve mice resistance to L. monocytogenes and RMA-S lymphoma challenge. In FIG. 11a, wild type (wt) B6 mice pretreated with CD137 mAb or control antibody were infected i.p. with 1×10⁶ L. monocytogenes. CFU in liver was shown day 2 after infection. Viability was checked daily for ten days (n=7 each group). The results are from one representative of at least two independent experiments with similar results. In FIG. 11b B6 IFN-γ KO transferred with or without 2×10⁷ purified wt T cell were pretreated with CD137 mAb or control antibody in day −7 and −5. Mice were infected i.p. with 5×10⁵ L. monocytogenes and CPU in liver were checked 2 days after infection (n=4 each group). In FIG. 11c, B6 IFNγ KO mice transferred with 2×10⁶ purified memory OT-1 were pretreated with CD137 mAb or control mAb as describe in b, above. Mice were infected i.p. with L. monocytogenes. Bacterial titers in the liver were calculated 2 days after infection (n=4 each group). 1×10⁶ CFSE-labeled RMA-S tumor cells were injected i.p. into wt B6 (FIGS. 11d, e) or RAG-1 KO (FIG. 11) mice pretreated with 2A mAb (CD137 mAb) or control mAb. Peritoneal cells were collected at 24 and 48 hours later, counted and stained with propidium iodide (PI). The percentage of RMA-S cell in total peritoneal cell was indicated by staining of CFSE and analyzed using flow cytometry (FIG. 11d). The total number of peritoneal RMA-S cell harvested is also shown (FIGS. 11e, f). The results are from one representative of two independent experiments with three mice each. **, p<0.001, CD137 mAb treated versus control mAb.

FIG. 12 shows the role of NK1.1+ cells in CD137-stimulated innate immunity against RMA-S tumors. B6 mice were treated with 2A mAb or Rat Ig control mAb on day 0 and day 2. Two additional groups were injected with 0.25 mg of PK136 (anti-NK1.1 depletion mAb) on day −2, day 0 and day 3 to deplete NK1.1+ cells. On day 7, mice were challenged i.p. with 1×10⁶ CFSE-labeled RMA-S tumor cells. Peritoneal cells were collected at 24 hours, counted and stained with propidium iodide (PI). FIG. 12 consists of four panels showing the percentage of RMA-S cells in total PI-negative peritoneal cells, as indicated by staining of CFSE using flow cytometry. Data shown are from one representative of three mice in each group.

### DETAILED DESCRIPTION OF THE INVENTION

The invention features compositions and methods that are useful for increasing immune function. Such methods can be employed to enhance innate immunity for the prevention or treatment of pathogen infections (e.g., bacterial, viral, or fungal infections), or cancer. In particular, the invention provides antibodies that specifically bind a CD137 antigen on the surface of T lymphocytes. The invention is based, at least in part on the observation that stimulation of CD137 on memory T cells by agonist mAb was unexpectedly found to induce a potent, antigen-independent signal that increased the proliferation of memory T-cells. In addition, CD137 stimulation of memory T cells lead to an increase in the acquisition of innate immunity in naïve mice infected with Listeria monocytogenes and challenged with RMA-S lymphoma.

### CD137

CD137 (also known as ILA, 4-1BB, and TNFSFR9) is an inducible receptor of the tumor necrosis factor (TNF) receptor superfamily. CD137 is a 255-amino acid protein with 3 cysteine-rich motifs in the extracellular domain (characteristic of this receptor family), a transmembrane region, and a short N-terminal cytoplasmic portion containing potential phosphorylation sites. Its mouse homolog, 4-1BB, was cloned by Kwon and Weissman (1989) in screens for receptors expressed on activated lymphocytes and has 59.6% amino acid identity to ILA. Expression in primary cells is strictly activation dependent. Constitutive expression was detected only in oncogenically or virally transformed cells.

CD137 is expressed by activated T cells, NK cells, monocytes and dendritic cells (Chen 2002; Croft 2003), as well as other non-hematopoietic cells (Watts 2005). Its natural ligand, CD137L, is constitutively expressed on a fraction of dendritic cells, and is inducible on macrophage, B cells and T cells (Watts 2005). CD137 costimulation of naïve T cells in the presence of T cell receptor (TCR) engagement induces a broad spectrum of immunological functions, including T cell expansion, cytokine production and prevention of activation-induced death of effector T cells (Watts 2005). Recent studies demonstrate that the CD137 signal is also critical in the prevention and reversal of established CD8+ T cell tolerance and anergy in vivo (Wilcox et al., 2004). Agonistic CD137 monoclonal antibodies (mAb) are found to augment T cell-mediated immune responses against cancer and viral infection in animal models (Halstead et al., 2002; Wilcox et al., 2002a; Zhu and Chen 2003). In addition, the same mAbs are also effective in ameliorating autoimmune diseases in experimental animal models. The mechanism underlying these observations is not yet fully understood (Foell et al., 2003; Fukushima et al., 2005; Kim et al., 2005; Seo et al., 2004; Sun et al., 2002). A CD137 signal is also found to regulate altitude of memory T cell responses in antigen priming (Hendriks et al., 2005). In the absence of CD137L, primary T cell responses to viral antigens remain normal, however, the recall of memory CD8+ T cell responses is impaired (Bertram et al., 2002; Bertram et al., 2004; Tan et al., 1999). In addition to its effect in T cells, CD137 could also deliver a stimulatory signal to NK cells and dendritic cells (DC) to augment cytokine productions and antigen presentation function, respectively (Futagawa et al., 2002; Wilcox et al., 2002b; Wilcox et al., 2002c).

### Therapeutic and Prophylactic Methods

Agents identified as binding and/or stimulating a CD137 polypeptide are useful for preventing or ameliorating a disease associated with a deficiency in innate immunity or with a deficiency in the number or activity of memory T cells. Such deficiencies are often observed in patient's suffering from lymphopenic condition, including lymphopenia associated with a chronic infection or with chemotherapy. Diseases and disorders characterized by excess memory T cell death may be treated using the methods and compositions of the invention.

In one approach, an agent identified as described herein is administered to a patient identified as in need of an increase in innate immunity, identified as having lymphopenia, or identified as having a pathogen infection. In one embodiment, the therapeutic or prophylactic agent is administered systemically. In another embodiment, the therapeutic or prophylactic agent is administered to die site of a potential or actual disease-affected tissue. The dosage of the administered agent depends on a number of factors, including the size and health of the individual patient. For any particular subject, the specific dosage regimes should be adjusted over time according to the individual need and the professional judgment of the person administering or supervising the administration of the compositions.

### Screening Assays

The invention provides methods for enhancing an innate immune response by stimulating CD137. While the Examples described herein specifically discuss the use of the monoclonal antibody 2A described by Sun et al., Nat. Med. 2002 December; 8 (12):1405-13 and by Wilcox et al., J. Clin. Invest. 109, 651-659 (2002a), one skilled in the art understands that the methods of the invention are not so limited. Virtually any agent that specifically binds to CD137 or that stimulates CD138 may be employed in the methods of the invention.

Methods of the invention are useful for the high-throughput low-cost screening of candidate agents that increase an innate immune response. A candidate agent that specifically binds to CD137 and stimulates CD137 is then isolated and tested for activity in an in vitro assay or in vivo assay for its ability to induce memory T cell proliferation. One skilled in the art appreciates that the effects of a candidate agent on a cell is typically compared to a corresponding control cell not contacted with the candidate agent. Thus, the screening methods include comparing the proliferation of a memory T cell (or progenitor cell) contacted by a candidate agent to the proliferation of an untreated control cell.

In other embodiments, the expression or activity of CD137 in a cell treated with a candidate agent is compared to untreated control samples to identify a candidate compound that increases the expression or activity of CD137 in the contacted cell. Polypeptide expression or activity can be compared by procedures well known in the art, such as Western blotting, flow cytometry, immunocytochemistry, binding to magnetic and/or CD137-specific antibody-coated beads, in situ hybridization, fluorescence in situ hybridization (FISH), ELISA, microarray analysis, RT-PCR, Northern blotting, or calorimetric assays, such as the Bradford Assay and Lowry Assay.

In one working example, one or more candidate agents are added at varying concentrations to the culture medium containing a memory T cell. An agent that promotes the expression of a CD137 polypeptide expressed in the cell is considered useful in the invention; such an agent may be used, for example, as a therapeutic to prevent, delay, ameliorate, stabilize, or treat an injury, disease or disorder characterized by a deficiency in innate immunity or in a memory T cell or in the expression of a CD137 polypeptide produced by a human immune cell. Once identified, agents of the invention (e.g., agents that specifically bind to and/or stimulate CD137) may be used to increase an innate immune response in a patient in need thereof, or to increase memory T cell proliferation, such as the memory T cell proliferation in vitro. The memory T cell may be expanded in vitro and then administered to the patient. Alternatively, an agent identified according to a method of the invention is locally or systemically delivered to increase an innate immune response or increase T cell proliferation in situ.

If one embodiment, the effect of a candidate agent may, in the alternative, be measured at the level of CD137 polypeptide production using the same general approach and standard immunological techniques, such as Western blotting or immunoprecipitation with an antibody specific for CD137. For example, immunoassays may be used to detect or monitor the expression of CD137 in a memory T cell or other mammalian immunoresponsive cell. In one embodiment, the invention identifies a polyclonal or monoclonal antibody (produced as described herein) that is capable of binding to and activating a CD137 polypeptide. A compound that promotes an increase in the expression or activity of a CD137 polypeptide is considered particularly useful. Again, such a molecule may be used, for example, as a therapeutic to combat the pathogenicity of an infectious organism or to prevent or treat a neoplasia.

Alternatively, or in addition, candidate compounds may be identified by first assaying those that specifically bind to and activate a CD137 polypeptide of the invention and subsequently testing their effect on innate immunity or T cell proliferation as described in the Examples (e.g., using FACS analysis, LPS, Listeria monocytogenes or RMA-S lymphoma challenge). In one embodiment, the efficacy of a candidate agent is dependent upon its ability to interact with the CD137 polypeptide. Such an interaction can be readily assayed using any number of standard binding techniques and functional assays (e.g., those described in Ausubel et al., supra). For example, a candidate compound may be tested in vitro for interaction and binding with a polypeptide of the invention and its ability to modulate innate immunity, CD137 activation, or memory T cell proliferation may be assayed by any standard assays (e.g., those described herein). In one embodiment, division of T cells in spleens is determined by assaying BrdU incorporation using flow cytometry analysis. In another embodiment, kinetic analysis of memory T cell response to CD137 mAb is assayed by staining the cells with CD44 and CD122 (IL-2 receptor β) mAb to identify increases in CD44^{hi} T cells in CD4 and CD8 cell subsets.

Potential CD137 agonists or 2A mAb mimetics include its natural ligand (CD137 ligand), organic molecules, peptides, peptide mimetics, polypeptides, nucleic acid ligands, aptamers, and antibodies that bind to a CD137 polypeptide and stimulate its activity. Methods of assaying CD137 activation include assaying memory T cell proliferation, cytokine secretion and cytolytic activity for tumor cells. Potential agonists also include small molecules that bind to and activate the CD137 polypeptide.

In one particular example, a candidate compound that binds to a CD137 polypeptide may be identified using a chromatography-based technique. For example, a recombinant CD137 polypeptide of the invention may be purified by standard techniques from cells engineered to express the polypeptide, or may be chemically synthesized, once purified the peptide is immobilized on a column. A solution of candidate agents is then passed through the column, and an agent that specifically binds the CD137 polypeptide or a fragment thereof is identified on the basis of its ability to bind to CD137 polypeptide and to be immobilized on the column. To isolate the agent, the column is washed to remove non-specifically bound molecules, and the agent of interest is then released from the column and collected. Agents isolated by this method (or any other appropriate method) may, if desired, be further purified (e.g., by high performance liquid chromatography). In addition, these candidate agents may be tested for their ability to modulate innate immunity or memory T cell proliferation (e.g., as described herein). Agents isolated by this approach may also be used, for example, as therapeutics to treat or prevent the onset of a disease or disorder characterized by a reduction in innate immunity, to treat or prevent a neoplasia, or to treat or prevent a pathogen infection (e.g., bacteria, virus, or fungal infection). Compounds that are identified as binding to a CD137 polypeptide with an affinity constant less than or equal to 1 nM, 5 nM, 10 nM, 100 nM, 1 mM or 10 mM are considered particularly useful in the invention.

Such agents may be used, for example, as a therapeutic to combat the pathogenicity of an infectious pathogen. Optionally, agents identified in any of the above-described assays may be confirmed as useful in conferring protection against the development of a pathogen infection in any standard animal model (e.g., the LPS, Listeria monocytogenes or RMA-S lymphoma challenge) and, if successful, may be used as anti-pathogen therapeutics.

Each of the polynucleotide sequences provided herein may also be used in the discovery and development of antipathogenic compounds (e.g., antibiotics). The encoded CD137 protein, upon expression, can be used as a target for the screening of drugs to enhance innate immunity. The CD137 agonists of the invention may be employed, for instance, to inhibit and treat a variety of bacterial infections, including Listeria monocytogenes infection.

### Test Compounds and Extracts

In general, CD137 agonists (e.g., agents that specifically bind and stimulate a CD137 polypeptide) are identified from large libraries of natural product or synthetic (or semi-synthetic) extracts or chemical libraries or from polypeptide or nucleic acid libraries, according to methods known in the art. Those skilled in the field of drug discovery and development will understand that the precise source of test extracts or compounds is not critical to the screening procedure(s) of the invention. Agents used in screens may include known those known as therapeutics for the treatment of pathogen infections. Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods-known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al, Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al, Proc Natl Acad Sci USA 89:1865-1869, 1992) or on phage (Scott and Smith, Science 249:386-390, 1990; Devlin, Science 249:404-406, 1990; Cwirla et al. Proc. Nat. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have CD137 binding and/or stimulating activity further fractionation of the positive lead extract is necessary to isolate molecular constituents responsible for the observed effect. Thus, the goal of the extraction, fractionation, and purification process is the careful characterization and identification of a chemical entity within the crude extract that enhances innate immunity or that stimulates memory T cell proliferation. Methods of fractionation and purification of such heterogeneous extracts are known in the art. If desired, compounds shown to be useful as therapeutics are chemically modified according to methods known in the art.

### Pharmaceutical Therapeutics

The invention provides a simple means for identifying compositions (including nucleic acids, peptides, small molecule inhibitors, and 2A monoclonal antibody mimetics) capable of binding to an activating CD137, enhancing innate immunity, increasing memory T cell proliferation, or acting as therapeutics for the treatment or prevention of a neoplasia or a pathogen infection (e.g., bacterial, viral, or fungal infection). Accordingly, a chemical entity discovered to have medicinal value using the methods described herein is useful as a drug or as information for structural modification of existing compounds, e.g., by rational drug design. Such methods are useful for screening agents having an effect on a variety of conditions characterized by a reduction in innate immunity.

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remigton's Pharmaceutical Sciences by E. W. Martin. The amount of the therapeutic agent to be administered varies depending upon the manner of administration, the age and body weight of the patient, and with the clinical symptoms of the pathogen infection or neoplasia. Generally, amounts will be in the range of those used for other agents used in the treatment of other diseases associated with pathogen infection or neoplasia, although in certain instances lower amounts will be needed because of the increased specificity of the compound. A compound is administered at a dosage that activates CD137 or that increases memory T cell proliferation as determined by a method known to one skilled in the art, or using any that assay that measures the expression or the biological activity of a CD137 polypeptide.

### Recombinant Polypeptide Expression

The invention provides recombinant CD137 polypeptides that may be used to induce antibody formation in a suitable host. Recombinant CD137 polypeptides of the invention are produced using virtually any method known to the skilled artisan. Typically, recombinant polypeptides are produced by transformation of a suitable host cell with all or part of a polypeptide-encoding nucleic acid molecule or fragment thereof in a suitable expression vehicle.

Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., Current Protocol in Molecular Biology, New York: John Wiley and Sons, 1997). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (e.g., pET-28) (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Alternatively, recombinant CD137 polypeptides of the invention are expressed in Pichia pastoris, a methylotrophic yeast. Pichia is capable of metabolizing methanol as the sole carbon source. The first step in the metabolism of methanol is the oxidation of methanol to formaldehyde by the enzyme, alcohol oxidase. Expression of this enzyme, which is coded for by the AOX1 gene is induced by methanol. The AOX1 promoter can be used for inducible polypeptide expression or the GAP promoter for constitutive expression of a gene of interest.

Once the recombinant CD137 polypeptide of the invention is expressed, it is isolated, for example, using affinity chromatography. In one example, an antibody (e.g., produced as described herein) raised against a polypeptide of the invention may be attached to a column and used to isolate the recombinant CD137 polypeptide. Lysis and fractionation of polypeptide-harboring cells prior to affinity chromatography may be performed by standard methods (see, e.g., Ausubel et al., supra). Alternatively, the polypeptide is isolated using a sequence tag, such as a hexahistidine tag, that binds to nickel column.

Once isolated, the recombinant CD137 polypeptide can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

### CD137 Polypeptides and Analogs

Also included in the invention are transCD137 polypeptides or fragments thereof that are modified in ways that enhance their ability to act as antigens to induce the production of agonistic antibodies. The invention provides methods for optimizing CD137 amino acid sequence or nucleic acid sequence by producing an alteration in the sequence. Such alterations may include certain mutations, deletions, insertions, or post-translational modifications. The invention further includes analogs of any naturally-occurring CD137 polypeptide of the invention. Analogs can differ from a naturally-occurring polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino, acid sequence of the invention. The length of sequence comparison is at least 5, 10, 15 or 20 amino acid residues, preferably at least 25, 50, or 75 amino acid residues, and more preferably more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., .beta. or .gamma. amino acids.

In addition to full-length polypeptides, the invention also includes fragments of any one of the polypeptides of the invention. As used herein, the term “a fragment” means at least 5, 10, 13, or 15. In other embodiments a fragment is at least 20 contiguous amino acids, at least 30 contiguous amino acids, or at least 50 contiguous amino acids, and in other embodiments at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

In one embodiment, the invention provides a 2A monoclonal antibody analogs having a chemical structure designed to mimic the CD137 binding and agonist activity of the 2A antibody. Such analogs are administered according to methods of the invention. 2A monoclonal antibody analogs may exceed the physiological activity of the original antibody. Methods of analog design are well known in the art, and synthesis of analogs can be carried out according to such methods by modifying the chemical structures such that the resultant analogs increase the reprogramming or regenerative activity of a reference transcription factor/protein transduction domain fusion polypeptide. These chemical modifications include, but are not limited to, substituting alternative R groups and varying the degree of saturation at specific carbon atoms of a reference polypeptide. Preferably, the analogs are relatively resistant to in vivo degradation, resulting in a more prolonged therapeutic effect upon administration. Assays for measuring functional activity include, but are not limited to, those described in the Examples below.

### Antibodies

Antibodies are well known to those of ordinary skill in the science of immunology. Particularly useful in the methods of the invention are antibodies that specifically bind a CD137 polypeptide that is expressed in memory T cell. As used herein, the term “antibody” means not only intact antibody molecules, but also fragments of antibody molecules that retain immunogen binding ability and act as CD137 mimetics to enhance innate immunity. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments which lack the Fc fragment of intact antibody, clear more rapidly from the circulation, and may have less non-specific tissue binding of an intact antibody (Wahl et al., J. Nucl. Med. 24:316-325 (1983). The antibodies of the invention comprise whole native antibodies, bispecific antibodies; chimeric antibodies; Fab, Fab′, single chain V region fragments (scFv) and fusion polypeptides.

In one embodiment, an antibody that binds a CD137 polypeptide is a monoclonal antibody agonist. Alternatively, the antibody is a polyclonal antibody agonist. The preparation and use of polyclonal antibodies are also known the skilled artisan. The invention also encompasses hybrid antibodies, in which one pair of heavy and light chains is obtained from a first antibody, while the other pair of heavy and light chains is obtained from a different second antibody. Such hybrids may also be formed using humanized heavy and light chains. Such antibodies are often referred to as “chimeric” antibodies.

In general, intact antibodies are said to contain “Fc” and “Fab” regions. The Fc regions are involved in complement activation and are not involved in antigen binding. An antibody from which the Fc′ region has been enzymatically cleaved, or which has been produced without the Fc′ region, designated an “F(ab′)₂” fragment, retains both of the antigen binding sites of the intact antibody. Similarly, an antibody from which the Fc region has been enzymatically cleaved, or which has been produced without the Fc region, designated an “Fab′” fragment, retains one of the antigen binding sites of the intact antibody. Fab′ fragments consist of a covalently bound antibody light chain and a portion of the antibody heavy chain, denoted “Fd.”The Fd fragments are the major determinants of antibody specificity (a single Fd fragment may be associated with up to ten different light chains without altering antibody specificity). Isolated Fd fragments retain the ability to specifically bind to immunogenic epitopes.

Antibodies can be made by any of the methods known in the art utilizing a CD137 polypeptide, or immunogenic fragments thereof, as an immunogen. One method of obtaining antibodies is to immunize suitable host animals with an immunogen and to follow standard procedures for polyclonal or monoclonal antibody production. The immunogen will facilitate presentation of the immunogen on the cell surface. Immunization of a suitable host can be carried out in a number of ways. Nucleic acid sequences encoding an CD137 polypeptide, or immunogenic fragments thereof, can be provided to the host in a delivery vehicle that is taken up by immune cells of the host. The cells will in turn express the receptor on the cell surface generating an immunogenic response in the host. Alternatively, nucleic acid sequences encoding a CD137 polypeptide, or immunogenic fragments thereof, can be expressed in cells in vitro, followed by isolation of the polypeptide and administration of the receptor to a suitable host in which antibodies are raised.

Using either approach, antibodies can then be purified from the host. Antibody purification methods may include salt precipitation (for example, with ammonium sulfate), ion exchange chromatography (for example, on a cationic or anionic exchange column preferably run at neutral pH and eluted with step gradients of increasing ionic strength), gel filtration chromatography (including gel filtration HPLC), and chromatography on affinity resins such as protein A, protein G, hydroxyapatite, and anti-immunoglobulin.

Antibodies can be conveniently produced from hybridoma cells engineered to express the antibody. Methods of making hybridomas are well known in the art. The hybridoma cells can be cultured in a suitable medium, and spent medium can be used as an antibody source. Polynucleotides encoding the antibody of interest can in turn be obtained from the hybridoma that produces the antibody, and then the antibody may be produced synthetically or recombinantly from these DNA sequences. For the production of large amounts of antibody, it is generally more convenient to obtain an ascites fluid. The method of raising ascites generally comprises injecting hybridoma cells into an immunologically naive histocompatible or immunotolerant mammal, especially a mouse. The mammal may be primed for ascites production by prior administration of a suitable composition; e.g., Pristane.

Monoclonal antibodies (Mabs) produced by methods of the invention can be “humanized” by methods known in the art. “Humanized” antibodies are antibodies in which at least part of the sequence has been altered from its initial form to render it more like human immunoglobulins. Techniques to humanize antibodies are particularly useful when non-human animal (e.g., murine) antibodies are generated. Examples of methods for humanizing a murine antibody are provided in U.S. Pat. Nos. 4,816,567, 5,530,101, 5,225,539, 5,585,089, 5,693,762 and 5,859,205. Once antibodies that specifically bind a CD137 polypeptide are identified, the antibodies ability to stimulate CD137 is assayed, for example, by assaying memory T cell proliferation.

Antibodies according to the invention may also be single chain antibodies. Single chain antibodies (“scFv”) refer to single polypeptide chain binding proteins having the characteristics and binding ability of multi chain variable regions of antibody molecules. Single chain V region fragments are made by linking L and/or H chain V regions by using a short linking peptide, as described in Bird et al. (1988) Science 242:423 426. Phage display of single chain Fv (scFv) offers a new way to produce monoclonal antibodies with defined binding specificities (Winter G, et al. 1994). In screening phage display libraries, for example, the phage express scFv fragments on the surface of their coat with a large variety of complementarity determining regions (CDRs). This technique is well known in the art. In particular embodiments, phage-displayed human antibody library are used to derive scFvs specific for CD137. A repertoire of many different scFvs can be displayed on the surface of filamentous bacteriophage, allowing phages with a specific antigen-binding activity to be selected by panning on the target antigen (Winter et al., supra). This approach has several advantages compared to the traditional hybridoma technology; (i) monoclonal antibodies can be isolated faster and without the need for animal immunization (Hoogenboom et. al. 1992); (ii) the use of a naïve library (derived from non-immunized donors) allows the selection of antibodies against self-antigen and weakly immunogenic proteins (Vaughan et. al. 1996); (iii) scFvs can be efficiently and economically produced in bacteria or in other expression systems (Miller et. al. 2005). ScFv antibodies contain the variable regions of heavy and light chains connected by a linker peptide and represent the smallest units retaining the antigen-binding specificity of whole IgGs (Bird et al., supra). Importantly, when these antibody fragments are of human origin, adverse immune responses in human therapy can be minimized (Laffly et. al. 2005).

### Formulation of Pharmaceutical Compositions

The administration of a compound for the treatment of a pathogen infection or neoplasia may be by any suitable means that results in a concentration of the therapeutic that, combined with other components, is effective in ameliorating, reducing, or stabilizing a pathogen infection or neoplasia. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a pathogen infection or neoplasia by using carriers or chemical derivatives to deliver the therapeutic agent to a particular cell type (e.g., memory T cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a pathogen infection or neoplasia, the composition may include suitable parenterally acceptable carriers and/or excipients. The active therapeutic agent(s) may be incorporated into microspheres, microcapsules; nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active anti-pathogen infection or anti-neoplasia therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Solid Dosage Forms For Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active a anti-pathogen or anti-neoplasia therapeutic substances. The coating may be applied on the solid dosage form in a similar manner as that described in Encyclopedia of Pharmaceutical Technology, supra.

At least two anti-pathogen or anti-neoplasia therapeutics may be mixed together in the tablet, or may be partitioned. In one example, the first active anti-pathogen or anti-neoplasia therapeutic is contained on the inside of the tablet, and the second active anti-pathogen or anti-neoplasia therapeutic is on the outside, such that a substantial portion of the second active anti-pathogen or anti-neoplasia therapeutic is released prior to the release of the first active anti-pathogen or anti-neoplasia therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

### Controlled Release Oral Dosage Forms

Controlled release compositions for oral use may, e.g., be constructed to release the active anti-pathogen or anti-neoplasia therapeutic by controlling the dissolution and/or the diffusion of the active substance. Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

### Combination Therapies

Optionally, anti-pathogen or anti-neoplasia therapeutic may be administered in combination with any other standard anti-pathogen or anti-neoplasia therapy; such methods are known to the skilled artisan and described in Remington's Pharmaceutical Sciences by E. W. Martin.

The invention provides kits for the treatment or prevention of a neoplasia, lymphopenia, or to treat a pathogen infection. In one embodiment, the kit includes a therapeutic or prophylactic composition containing an effective amount of an agent that specifically binds an stimulates a CD137 polypeptide in unit dosage form. In some embodiments, the kit comprises a sterile container which contains a therapeutic or prophylactic vaccine; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired an agent that specifically binds an stimulates a CD137 polypeptide (e.g., such as a 2A monoclonal antibody) is provided together with instructions for administering the agent to a subject having or at risk of developing a pathogen infection, lymphopenia, or neoplasia. The instructions will generally include information about the use of the composition for the treatment or prevention of pathogen infection, lymphopenia, or neoplasia. In other embodiments, the instructions include at least one of the following: description of the therapeutic agent; dosage schedule and administration for treatment or prevention of ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The present invention provides methods of treating subjects in need of increased innate immunity, as well as neoplastic diseases and/or pathogen infections or symptoms thereof which comprise administering a therapeutically effective amount of a pharmaceutical composition comprising a agent described herein to a subject (e.g., a mammal such as a human). Thus, one embodiment is a method of treating a subject suffering from or susceptible to a neoplastic diseases and/or pathogen infections or disorder or symptom thereof. The method includes the step of administering to the mammal a therapeutic amount of an amount of an agent herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as an agent of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The agents herein may be also used in the treatment of any other disorders in which a reduction in memory T cell number may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with neoplasia, pathogen infection, lymphopenia, or a decrease in memory T cell number, in which the subject has been administered a therapeutic amount of a therapeutic agent herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Knockout of CD137

The invention further provides mice having a “knockout” of the CD137 gene, which exhibits associated deficits in its innate immune response, and cells derived from such animals, which may be maintained in culture. An exemplary knockout mouse is described in the examples. Cells having reduced expression of a gene of interest are generated using any method known in the art. In one embodiment, a targeting vector is used that creates a knockout mutation in a CD137 gene. The targeting vector is introduced into a suitable cell (e.g., ES cell) or cell line to generate one or more cell lines that carry a knockout mutation. By a “knockout mutation” is meant an artificially-induced alteration in a nucleic acid molecule (created by recombinant DNA technology or deliberate exposure to a mutagen) that reduces the biological activity of the CD137 polypeptide normally encoded therefrom by at least about 50%, 75%, 80%, 90%, 95%, or more relative to the unmutated gene. The mutation can be, without limitation, an insertion, deletion, frameshift mutation, or a missense mutation. The targeting construct may result in the disruption of the gene of interest, e.g., by insertion of a heterologous sequence containing stop codons.

Gene targeting is a technique utilizing homologous recombination between an engineered exogenous DNA fragment and the genome of a mouse embryonic stem (ES) cell. Recombination between identical regions contained within the introduced DNA fragment and the native chromosome will lead to the replacement of a portion of the chromosome with the engineered DNA. These modified ES cells can then be injected into mouse blastocysts where they can incorporate and contribute to the fetal development along with the blastomeres from the ICM (inner cell mass). These techniques can be used to ablate (knockout) gene function throughout the mouse, in selected tissues, or at specific time points of mouse development. They can also be used to introduce mutations into the genome at a desired location. Essentially all gene targeting experiments have the following steps:
1. construction of targeting vector containing regions of identity with the mouse chromosome (homology units or arms), a selectable marker (generally a cassette that confers neomycin (G418) resistance) and planned modifications that ablate or alter the expression of the targeted gene or region of chromosome
2. introduction of the linearized targeting vector into mouse ES cells and selection and screening for those targeted ES clones that have integrated the planned modifications by homologous recombination
3. microinjection of targeted ES cells into blastocysts to generate mice chimeric for the targeted ES cells and host blastocyst cells
   If desired, the chimeric mouse is then bred to generate a mouse that is homozygous for the knockout. Such mice typically lack detectable levels of the targeted gene.

Other methods for gene knock out may be used. For example, FRT sequences may be introduced into the cell such that they flank the gene of interest. Transient or continuous expression of the FLP protein is then used to induce site-directed recombination, resulting in the excision of the gene of interest. The use of the FLP/FRT system is well established in the art and is described in, for example, U.S. Pat. No. 5,527,695, and in Lyznik et al. (Nucleic Acid Research 24:3784-3789, 1996).

Furthermore, the targeting construct may contain a sequence that allows for conditional expression of the gene of interest. For example, a sequence may be inserted into the gene of interest that results in the protein not being expressed in the presence of tetracycline. Such conditional expression of a gene is described in, for example, Yamamoto et al. (Cell 101:57-66, 2000)).

Conditional knockout cells are also produced using the Cre-lox recombination system. Cre is an enzyme that excises DNA between two recognition sites termed loxP. The cre transgene may be under the control of an inducible, developmentally regulated, tissue specific, or cell-type specific promoter. In the presence of Cre, the gene, for example a nucleic acid sequence described herein, flanked by loxP sites is excised, generating a knockout. This system is described, for example, in Kilby et al. (Trends in Genetics 9:413-421, 1993).

In one embodiment, the invention provides a rodent (e.g., a rat or mouse) having a reduction in the expression of a CD137 polypeptide. In addition, cell lines from these rodents may be established by methods standard in the art. Construction of knockout mutations can be accomplished using any suitable genetic engineering technique, such as those described in Ausubel et al. (Current Protocols in Molecular Biology, John Wiley & Sons, New York, 2000).

Animals suitable for knockout experiments can be obtained from standard commercial sources such as Taconic (Germantown, N.Y.). Many strains are suitable, but Swiss Webster (Taconic) female mice are desirable for embryo retrieval and transfer. B6D2F (Taconic) males can be used for mating and vasectomized Swiss Webster studs can be used to stimulate pseudopregnancy. Vasectomized mice and rats are publicly available from the above-mentioned suppliers. However, one skilled in the art would also know how to make a knockout mouse or rat. An example of a protocol that can be used to produce a knockout animal is provided below in the Examples.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

The invention will now be further illustrated with reference to the following Methods and Examples. It will be appreciated that what follows is by way of example only and that modifications to detail may be made while still falling within the scope of the invention.

### Example 1

### CD137 Agonist mAb Induces Proliferation of Memory, but not Naïve, T Cells in Naïve Mice

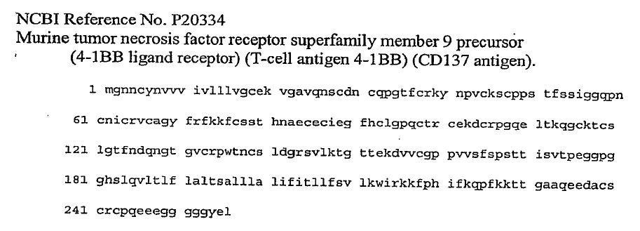

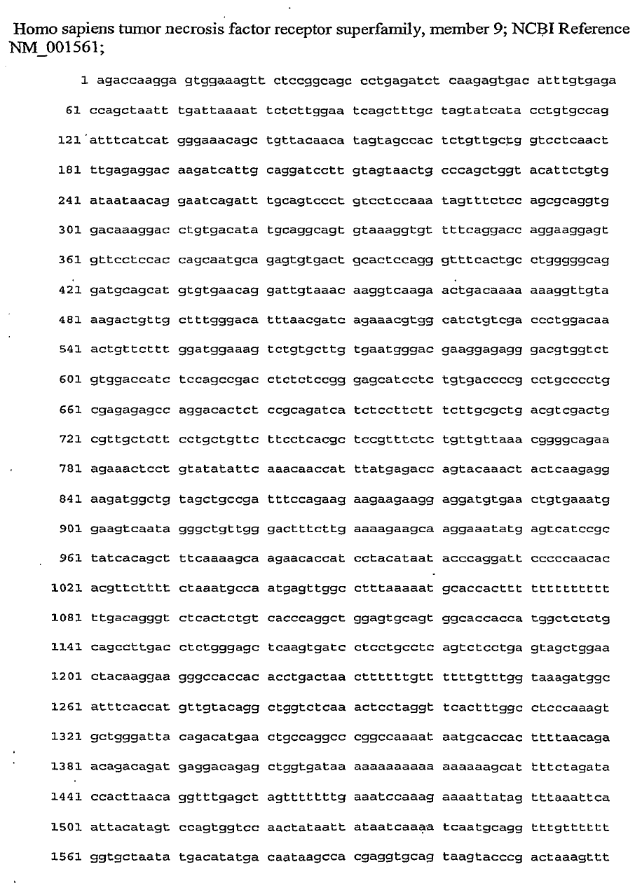

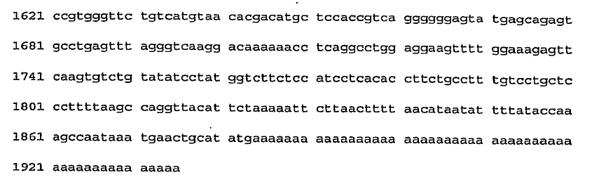

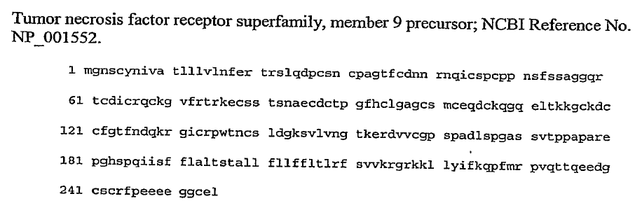

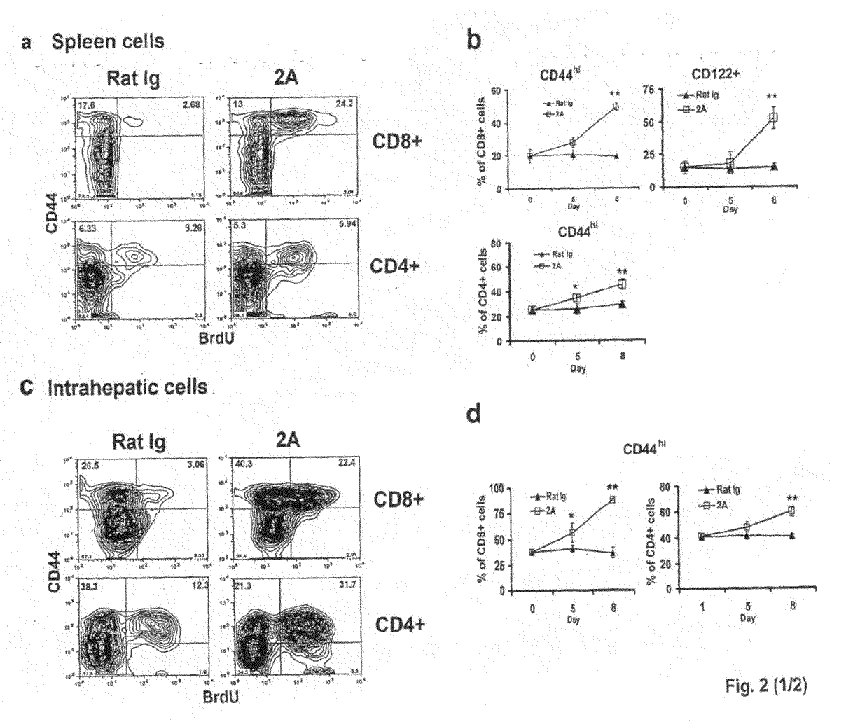

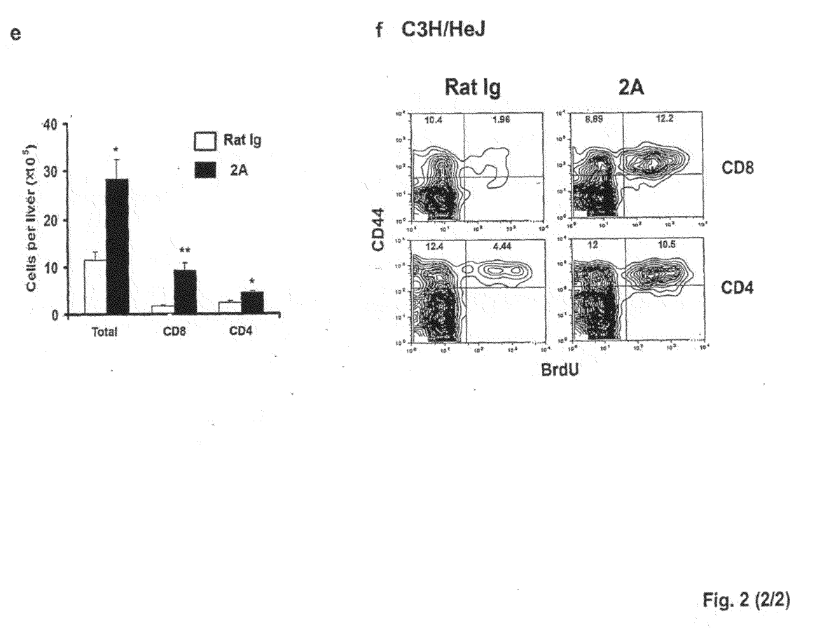

While the roles of CD137 in co-stimulating adaptive immunity have been studied extensively, the effects of CD137 in innate immunity have yet to be explored. Naïve B6 mice were injected with a CD137 agonist monoclonal antibody (mAb) (clone 2A; Wilcox et al., 2002a) and subsequently fed with BrdU in drinking water continuously for 4 days to mark dividing cells. Division of T cells in spleens was then determined by BrdU incorporation using flow cytometry analysis. While only a small fraction of CD44^{hi} cells (<5%) underwent division in control mAb-treated mice, CD137 mAb treatment induced >10 fold increases of CD44^{hi} cells which undergo division, as shown in FIG. 2a. This result was observed in both CD8+ and CD4+ T cells. The effect on CD8+ T cells was more profound. No significant change in T cell apoptosis was observed during 7 days of CD137 mAb treatment. Without wishing to be tied to theory, this suggests that the increase in the number of CD44^{hi} T cells was due to enhanced proliferation. In contrast, CD44^{lo} T cells did not show significant division, although a small increase in the number of dividing cells was observed in CD4 subset in some experiments (FIG. 2a).

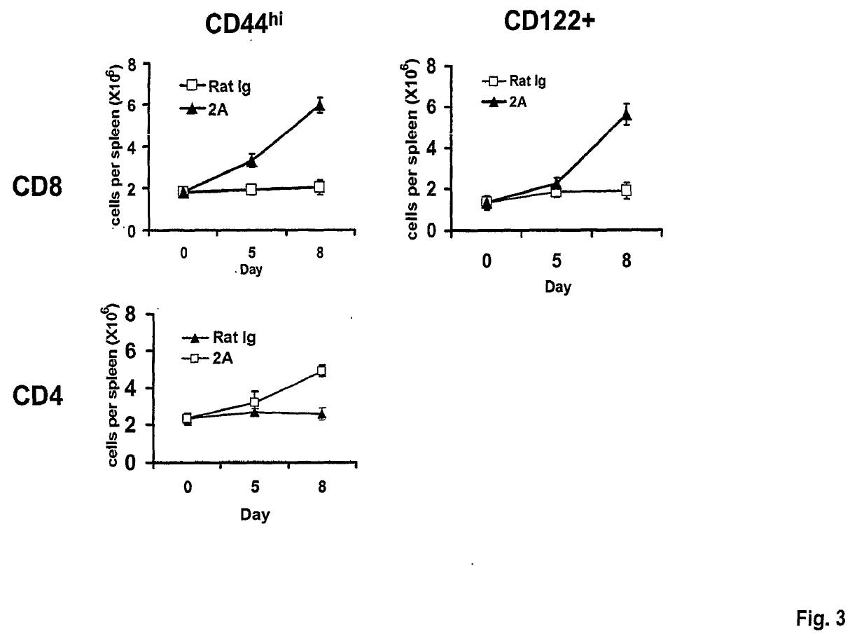

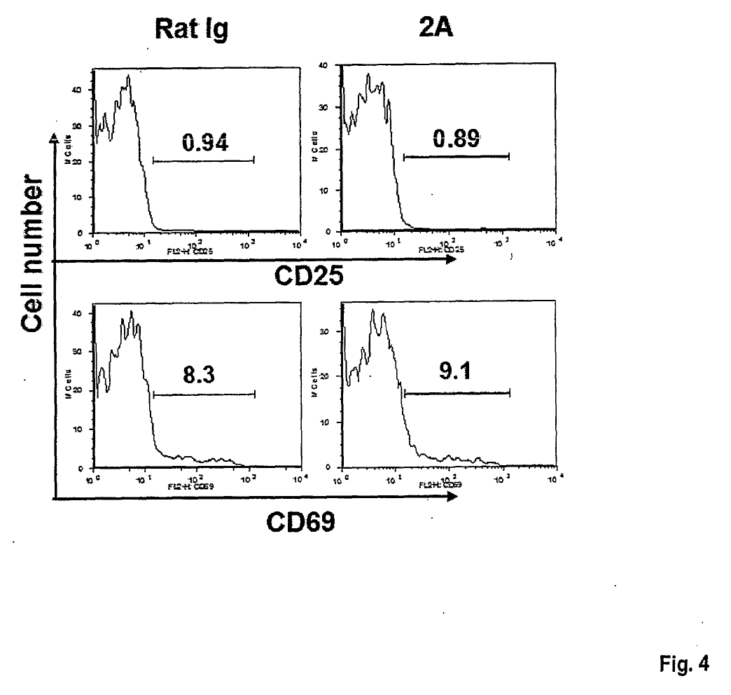

While two doses of the mAb were administered in initial experiments, additional studies demonstrate that a single injection of 2A CD137 mAb is sufficient to induce proliferation. In addition to the proliferation of spleen T cells, which are central memory T cells, injection of CD137 mAb also induced a significant increase in the number of intrahepatic CD44^{hi} T cells in both CD8+ and CD4+ subsets, as shown in FIG. 2c, indicating that CD137 stimulation is also effective in inducing the proliferation of effector memory T cells. Kinetic analysis of memory T cell proliferative response to CD137 mAb using both CD44 mAb and CD122 (IL-2 receptor β) mAb indicates that the number of CD44^{hi} T cells in both CD4 and CD8 subsets increased progressively. On day 8, the percentage (FIGS. 2b-2d) and absolute number (FIG. 3) of these cells in both spleens and livers reached more than double. CD137 mAb had only minimal effect in CD44^{lo} and CD122 negative cells, when employed in both the spleen and liver. In an analysis of intrahepatic T cells, both CD8+ and CD4+ subsets increased dramatically in number. More robust expansion was observed in the CD8+ subset, as shown in FIG. 2e. A similar effect was observed in the effect of the CD137 mAb on the proliferation of CD44^{hi} T cells in C3H/HeJ mice. This observation excludes the effect of lipopolysaccharides (LPS) (Tough et al. 1997), because this mouse strain is genetically defective at the Toll-like receptor-4 (TLR4) locus so as to be non-responsive to LPS (FIG. 2f). Moreover, as shown in FIG. 4, after CD137 mAb treatment there was no significant increase in either CD25 or CD69, which are markers for activation of T cells after TCR signaling. Thus, one measure of “T cell receptor signalling” is CD69 and CD25 upregulation. Taken together, this data indicates that both central and effector memory T cells respond to CD137 stimulation.

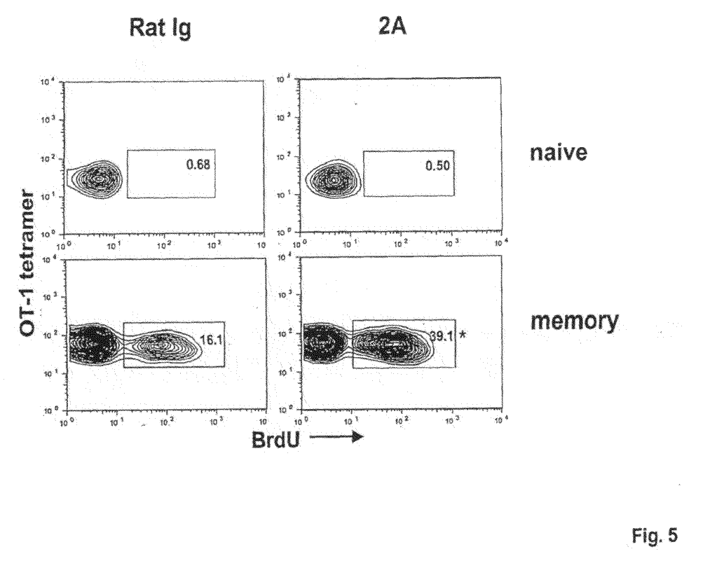

The above experiments support the rationale that CD44^{hi} memory-like T cells selectively expand upon CD137 mAb stimulation. Another possibility is that increased CD44^{hi} may represent an acquired phenotype of naïve T cells upon CD137 mAb treatment. To exclude this possibility, an adoptive transfer system was used in which naive OT-1 T cell Receptor (TCR) transgenic T cells are transferred into naïve B6 mice and monitored by specific tetramer for their responses to CD137 mAb. First, this strain was backcrossed to a RAG-1 KO background to guarantee that all T cells are naïve (CD44^{lo} and CD122-negative). As shown in the upper panel of FIG. 5, CD137 mAb treatment did not increase the incorporation of BrdU into transferred OT-1 cells as detected by the OT-1 tetramer. BrdU incorporation was used as an internal positive control. Significant increase of BrdU incorporation into OT-1 tetramer negative cells was observed, presumably due to expansion of memory T cells of recipient-origin. These results thus indicate that naïve T cells do not respond to CD137 mAb stimulation in vivo.

It was next examined whether memory OT-1 T cells could respond to CD137 stimulation in vivo. Purified OT-1 T cells were first stimulated in vitro by irradiated CD80/EG7, an EL4 mouse thymoma subline that expresses chicken OVA and murine CD80 co-stimulatory molecules in the presence of IL-2. This stimulation leads to activation of nearly 100% OT-1 cells as indicated by the expression of cell surface markers including CD44, CD69 and CD25. Activated T cells were purified and transferred into naive B6 mice to allow the generation of memory T cells (Bathe et al., 2001). Forty days later, flow cytometry analysis was performed. The flow cytometric analysis demonstrated that all OT-1 cells in spleens express high levels of CD44, and that >80% of these cells also express CD62 ligand (CD62L), thus indicating that they were mainly central memory T cells. As shown in the lower panel of FIG. 5, CD137 mAb treatment induced significantly higher levels of cell division in OT-1 memory cells than did treatment with control mAb (39.1% vs. 16.1%).

Taken together, this data shows that the agonist CD137 mAb, a mAb previously showed to be co-stimulatory for primed T cells (Wilcox et al., 2002a), does not activate naïve T cells when administered into naïve mice. These results provide direct evidence that CD137 engagement selectively triggers memory, but not naïve, T cell proliferation in vivo.

### Example 2

### CD137 on T Cells is Required for CD137 mAb-Induced Memory T Cell Proliferation

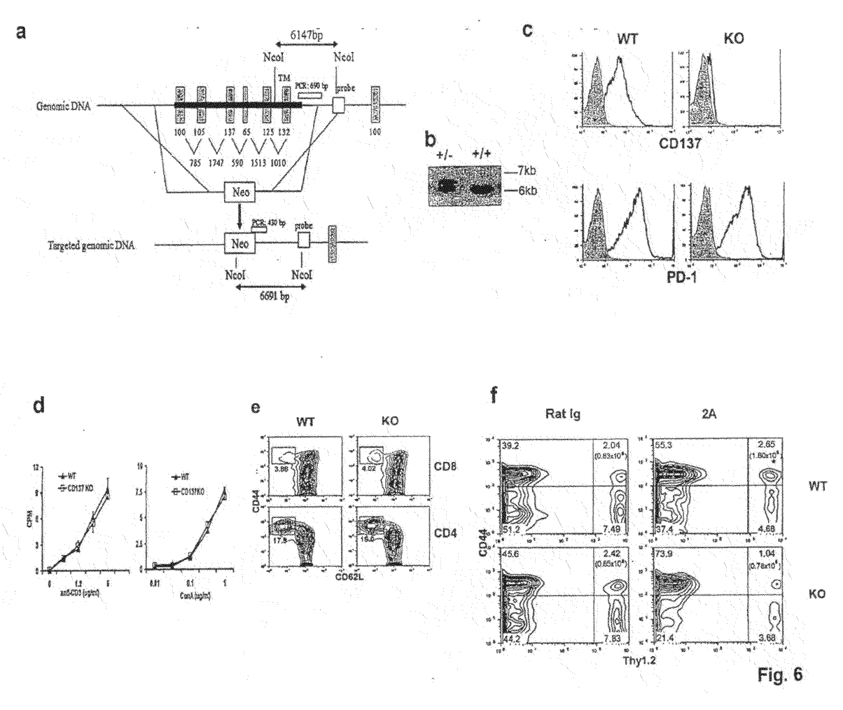

Subsequently, the effects of CD137 expression on memory T cells as a requirement for the effect of CD137 mAb were examined. A CD137 knockout (KO) mouse was generated by homologous recombination in 129 embryonic stem (ES) cells. A gene-targeting vector replaced exon 1-6 in the endogenous CD137 allele with a Neo-resistance cassette, thereby deleting the sequences encoding the signal peptide, the entire extracellular and transmembrane region of mouse CD137, as shown in FIG. 6a. Prior to being used in this study, mice were bred to the B6 background and further backcrossed for at least five generations. Southern blot analysis demonstrated the deletion of genomic DNA of CD137, shown in FIG. 6b. To confirm the absence of the CD137 protein in these mice, concanavalin A-activated (ConA) spleen cells from CD137 KO mice were stained with CD137 mAb. The results are shown in FIG. 6c. As expected, CD137 was detected in wild type (WT) but not KO T-cells. KO T-cells expressed normal levels of PD-1 molecule, a cell surface T cell activation marker (Okazaki et al., 2002).

CD137 KO mice have normal numbers and ratios of CD4+CD8+ double positive, CD4+, and CD8+ single positive T cells in the thymus. T cell populations in the spleens and lymph nodes also appear normal. These results are consistent to a previously published report (Kwon et al., 2002), indicating that CD137 signal does not affect T cell development in lymphoid organs. Activation of T cells from the KO mice by Con A or anti-CD3 mAb did not result in any significantly altered proliferation, as shown in FIG. 6d. There was also no evidence of autonomous activation of T cells, as seen by normal levels of CD62L and CD44 on CD4+ and CD8+ T cells in the KO mice (FIG. 6e). These results indicate that CD137 deficiency does not affect development and polyclonal activation of T cells.

Next, to determine the role of T cell-associated CD137, purified T cells from WT or CD137 KO mice (Thy1.2+) were transferred into congenic Thy1.1 B6 mice. The mice were subsequently treated with CD137 mAb (2A mAb). In this system, numbers of donor T cells could be specifically traced by anti-Thy1.2 mAb. CD137 mAb treatment did not increase the % of CD137KO donor CD44^{hi} cells in total CD8+ T cells (from 2.42% to 1.04%), as shown in FIG. 6f, lower panels. While this data indicates that the effect of CD137 mAb in the expansion of memory T cells was completely eliminated, the absolute numbers of donor CD44^{hi} cells remain the same: 0.85×10⁵ in control mAb-treated mice vs. 0.78×10⁵ in CD137 mAb-treated mice. The ratio change of donor cells from 2.42% to 1.04% is largely due to vigorous expansion of recipient CD44^{hi} cells (from 45.6% to 73.9%), leading to dilution of donor cells. In fact, the ratios of CD44^{hi} vs. CD44^{lo} donor cells were not affected by CD137 mAb treatment (2.42/7.83=0.31 vs. 1.04/3.68=0.28). In contrast, CD137 mAb treatment increased absolute number of CD137+/+ donor CD44^{hi} cells in spleens from 0.83×10⁵ to 1.80×10⁵ cells. This represents a 2.2 fold increase over the background. Taken together, these results demonstrate that CD137 on T cells are required for the effect from the mAb. Consistent with this, the ratios of CD44^{hi} vs. CD44^{lo} donor cells were increased significantly upon CD137 mAb treatment (2.04/7.49=0.27 vs. 2.65/4.68=0.57).

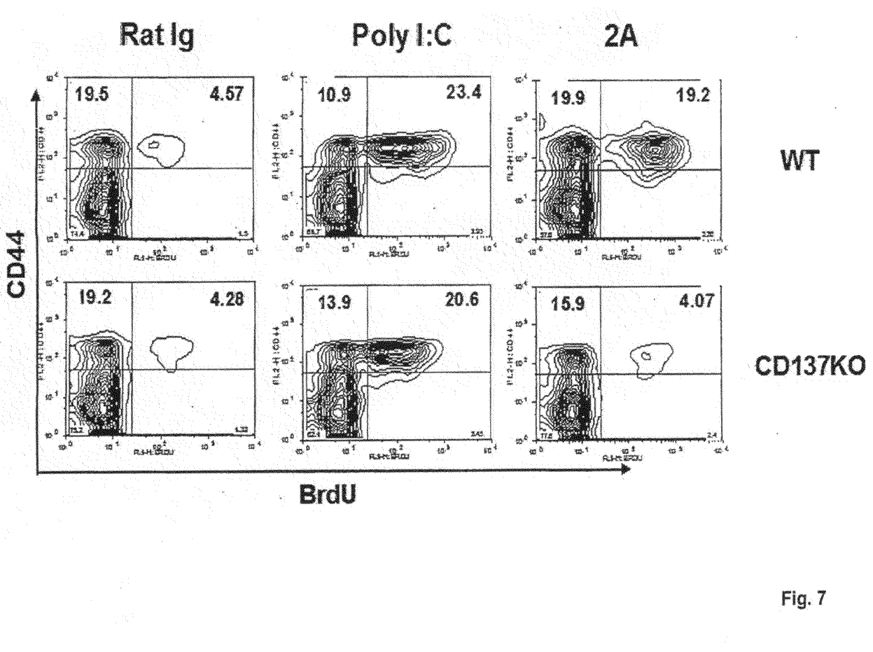

The failure of CD137-deficient memory T cells to respond to CD137 mAb was not caused by intrinsic defect because CD137KO memory T cells responded normally to poly I:C, a potent inducer of interleukin-15 (IL-15) growth factor for CD8+ memory T cells. While injection of 2A mAb induces vigorous proliferation of CD44^{hi} memory T cells, similar treatment did not have the same effect in CD137KO mice, as shown in FIG. 7.

Together, the results presented here thus support the idea that T cell-associated CD137 is mediates the effect of CD137 mAb on memory T cell expansion.

### Example 3

### CD137 Stimulation Promotes Homeostatic Proliferation of T Cells

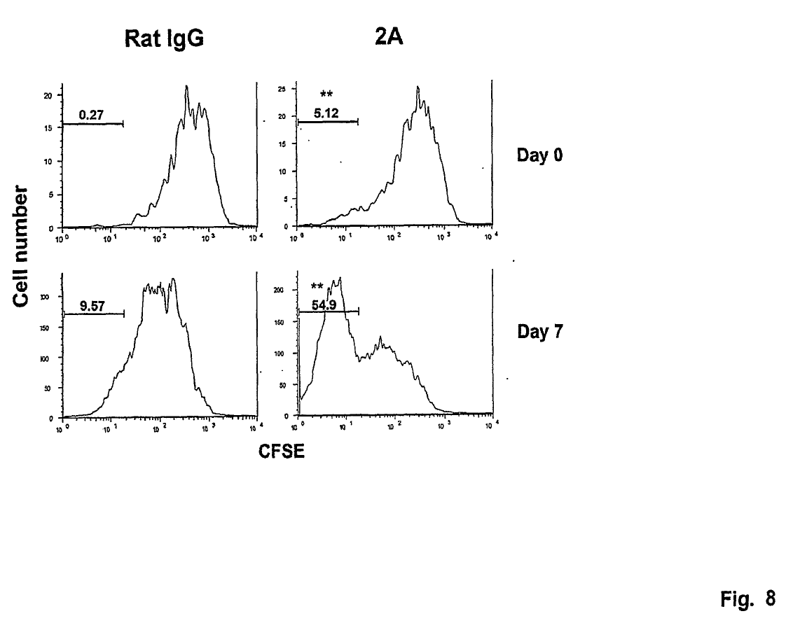

Naïve T cells, upon transfer into lymphopenic mice, will increase division and acquire memory T cell phenotypes, a phenomenon called homeostatic proliferation (Cho et al., 2000; Goldrath et al., 2000; Kieper and Jameson 1999; Murali-Krishna and Ahmed 2000). To test whether CD137 stimulation also promotes this process of homeostatic proliferation, 10⁶CFSE-labeled OT-1 x RAG-1KO T cells were transferred into sublethally irradiated B6 mice and treated with CD137 mAb on the same day. On day 6 after treatment, spleen cells were harvested and CFSE dilution/cell division was examined by flow cytometry. The majority of transferred OT-1 had divided more than one cycle and the pattern of cell division between the CD137 mAb-treated group and the control group was similar overall, as shown in the upper panel of FIG. 4. Interestingly, CD137 mAb-treated mice consistently contained a small population of OT-1 cells, which underwent more than five divisions (FIG. 8, upper panel). Consistent with previously published data (Cho et al., 2000; Goldrath et al., 2000), the levels of memory T cell markers CD44 and CD122 increased progressively over cell divisions, and only those cells experiencing more than five cell divisions acquired clear memory T cell markers (CD44^{hi}CD122+). It is thus possible that CD137 mAb might be effective only on those T cells which acquire a memory phenotype. Based on this observation, treatment with CD137 mAb was delayed until day 7 after OT-1 transfer. Cell division was examined 6 days later. After 6 days, more than 50% of transferred OT-1 cells underwent more than 7 divisions when treated with CD137 mAb. In contrast, in the control mice, only ˜10% of OT-1 T cells had undergone more than 7 divisions, as shown in FIG. 8, lower panel.

In addition to memory T cells in spleen and lymph nodes, memory T cells in liver also proliferate vigorously in response to CD137 mAb, indicating that both central and effector memory T cells could respond to CD137 signaling. Recently it has been shown that homeostatic proliferation in the hosts with lymphopenia triggers naive T cells to acquire the phenotypic and functional properties of memory cells without transition through the typical effector intermediates (Cho et al., 2000; Goldrath et al., 2000; Kieper & Jameson 1999; Murali-Krishna & Ahmed 2000). Consistent with this observation, a small number of naïve OT-1 cells transferred into sublethally-irradiated mice increased CD44 and CD122 expression, indicative of the memory phenotype, but not CD25 and CD69, indicative of the activation phenotype. Injection of CD137 mAb together with naïve T cell transfer had only minimal effect on cell division. However, on day 7 as most of transferred T cells divide and acquire phenotypes of memory T cells, CD137 mAb treatment clearly promotes T cell division, as shown in FIG. 8. These findings support that all types of memory T cells share common pathways to respond to CD137 stimulation for growth.

Taken together, these results suggest that CD137 mAb preferentially promotes proliferation of T cells which acquire memory phenotype during homeostatic proliferation.

### Example 4

### Effect of CD137 Stimulation on Memory T Cell Proliferation is Independent of MHC, IL-15 and IFN-γ

As shown previously in FIG. 5, CD137 mAb could drive proliferation of memory OT-1 T cells without supply of OVA antigen. While it could be interpreted from this data that CD137-triggered proliferation of memory T cells is independent of antigen, an alternative interpretation is that CD137 mAb-induced proliferation of memory T cells is still dependent on TCR interaction with MHC/self antigen, which could cross-react with OT-1 TCR. To exclude this possibility, the effect of CD137 mAb in H-2 Kb KO mice after transfer of memory OT-1 T cells, which is H-2 Kb-restricted, was tested. As previously shown, memory T cells were first generated by transfer of in vitro fully-activated OT-1 cells into naïve B6 mice for more than 40 days to generate memory T cells. CD8+ T cells were purified (>95%) by negative selection using MACS-bead. The cells were then CFSE-labeled and adoptively transferred into H-2 Kb KO mice, and then followed with CD137 mAb or control mAb treatment. Cell division of OT-1 memory cells was traced on day 7 by triple staining of CD8, OT-1 tetramer and CFSE. Memory OT-1 cells in CD137 mAb-treated mice has significant more cell division than that by control mAb (19.8% versus 7.49%), as shown by the dilution of the CFSE intensity (FIG. 9a). This result suggests that the effect of CD137 mAb does not require MHC recognition. To further validate this finding, the effect of an H-2 Kb blocking mAb (clone AF6.88.5) was tested in CD137 mAb-induced proliferation of memory T cells. This H-2 Kb-specific mAb could efficiently inhibit naïve OT-1 T cell homeostasis in lymphopenic B6 mice, which is believed to be a self MHC-dependent process (Jameson 2002). The treatment by CD137 mAb of B6 mice, which were transferred with memory OT-1 T cells, led to clearly increased BrdU incorporation, as shown in FIG. 9b.

Taken together, these results indicate that CD137 stimulation triggers memory T cell division in a self MHC-independent fashion, and the interaction between TCR and MHC is not required for CD137-induced proliferation of memory T cells.

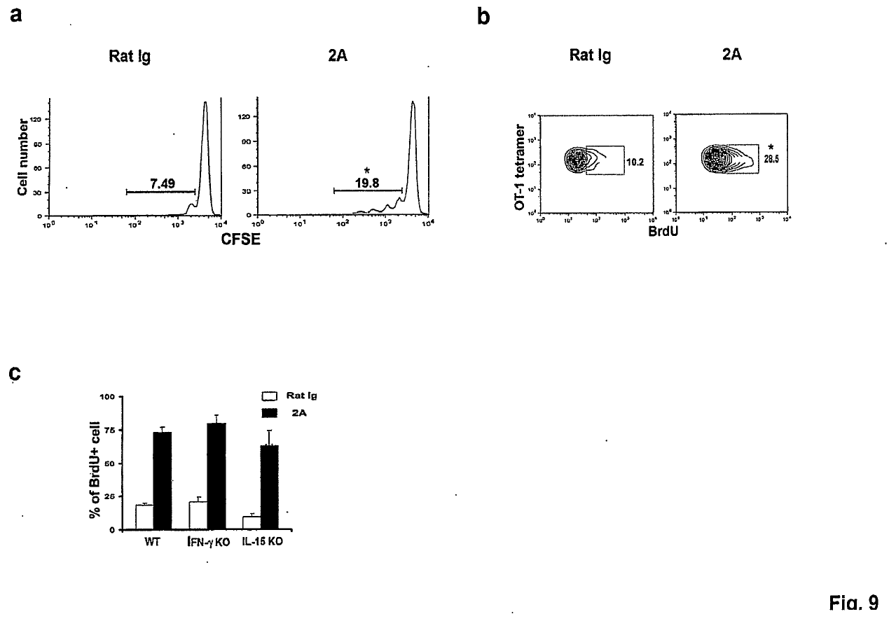

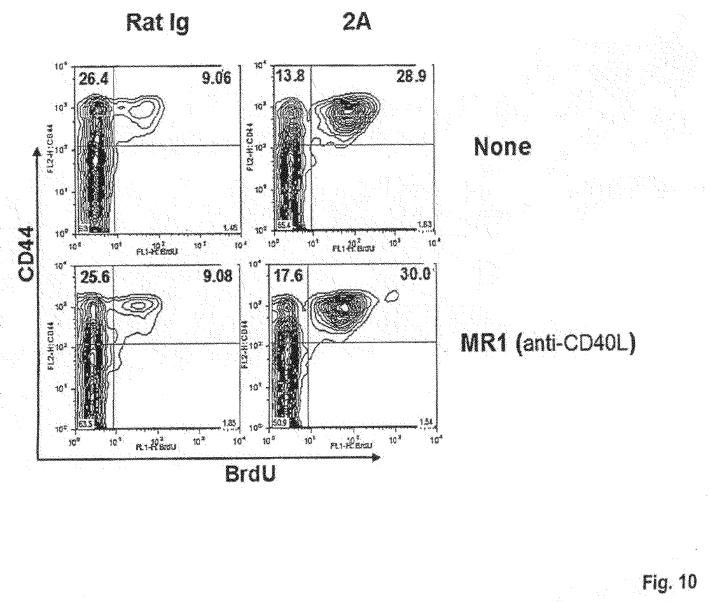

IL-15 is an important cytokine for the proliferation of CD8+ memory T cells (Becker et al., 2002; Zhang et al., 1998). Thus, it is possible that the effect of CD137 mAb is mediated by production of IL-15. In addition, CD137 mAb was found to induce IFN-γ secretion upon engagement of T cells in the presence of TCR signal, and it has been reported that the majority of CD137 mAb effects on T cell responses are dependent on IFN-γ (Watts 2005). To exclude these possibilities, IL-15 KO and IFN-γ KO mice were treated with CD137 mAb and subsequently fed with BrdU as described previously. Six days after treatment, CD8+ CD44^{hi} cells were gated, and the number of BrdU-positive cells was calculated. As shown in FIG. 9c, CD137 mAb stimulated memory T cell proliferation at comparable levels to that of control mAb in both IL-15 and IFN-γ KO mice. These results indicate that both IL-15 and IFN-γ are not required for CD137 mAb-triggered memory T cell proliferation. The results demonstrate that CD40 and CD40 ligand (CD40L) interaction is not required for the effect of CD137 mAb in memory T cells because inoculation of MR1 mAb, which is neutralizing mAb specific for mouse CD40L, does not affect the function of CD137 mAb, as shown in FIG. 10.

Further, the results show that stimulation of memory T cells by CD137 mAb in vivo does not increase the expression of CD69 and CD25, which are rapidly upregulated after TCR signaling (FIG. 4). These results thus suggest that CD137 mAb induces a distinct signal pathway on memory T cells. Taken together, the data presented herein may provide a unique opportunity to examine CD137 signal without interference by TCR signaling. IL-15, a cytokine critical for the proliferation of memory T cells, does not seem to be responsible for the effect because a comparable memory T cell proliferation to CD137 mAb treatment was also observed in IL-15 KO mice, as shown in FIG. 5.

### Example 5

### CD137 mAb Stimulates a Memory T Cell-Mediated and IFN-γ-Dependent Innate Immunity Against Listeria monocytogenes (LM) and RMA-S Lymphoma in Naïve Mice

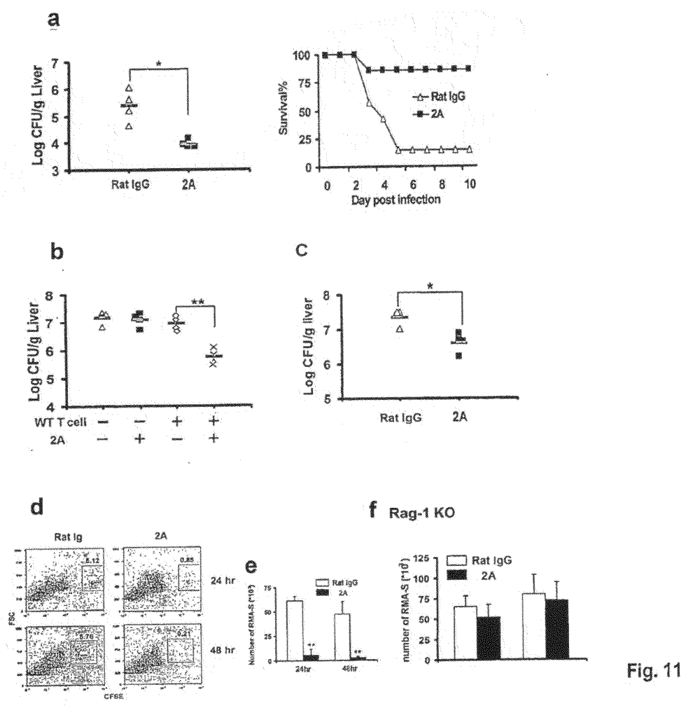

The data presented herein has shown that CD137 ligation by mAb is able to deliver a potent signal for growth of memory T cells. However, functional consequences of this effect are not known. It was first examined whether CD137 mAb was able to induce activation of the immune system. To test this, naive B6 mice were inoculated intraperitoneally (i.p.) with CD137 mAb on day 0 and day 2. At day 7, mice were challenged i.p. with 1×10⁶ CFU LM, a lethal dose for B6 mice. Forty-eight hours after infection, the livers from the mice that were pretreated with CD137 mAb demonstrated more than one log fewer bacteria than those treated with control mAb, as shown in the left panel graph of FIG. 11a. This rapid response indicates that the resistance to bacterial infection is mediated by innate, but not adaptive, immunity. Moreover, the majority of the mice that were treated by CD137 mAb survived, whereas nearly all mice that were treated by control mAb died within 6 days after challenge, as shown in the right panel graph of FIG. 11a.

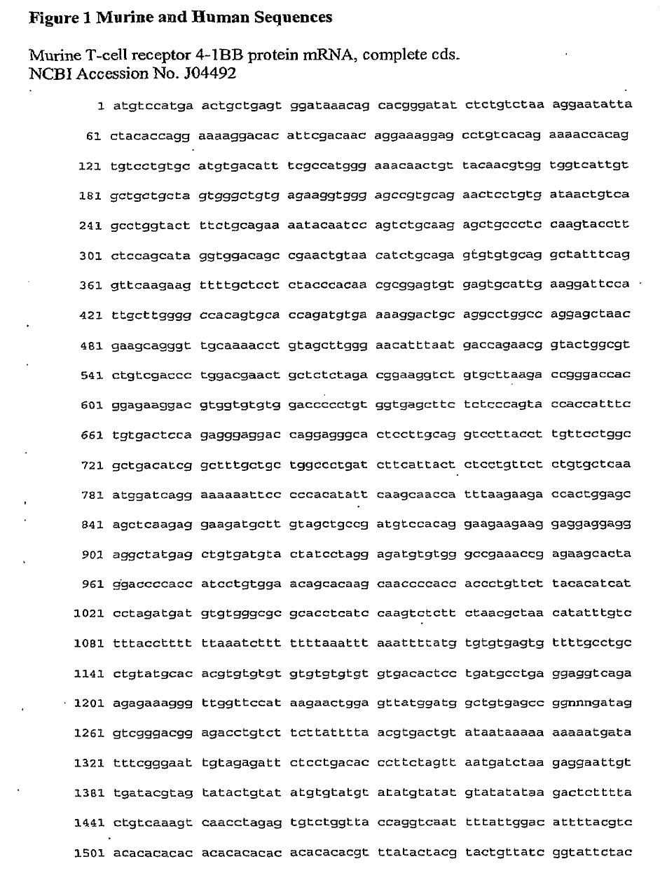

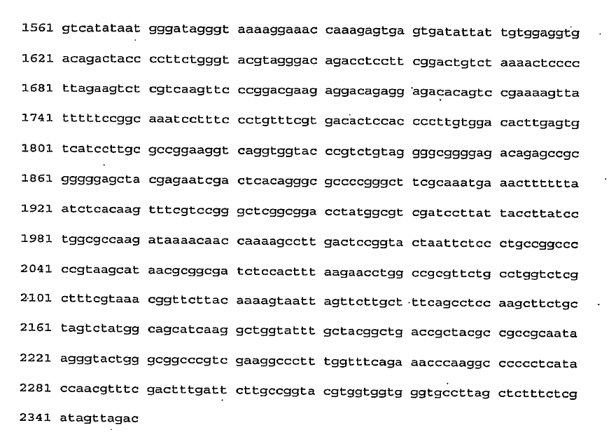

Next, the requirement of IFN-γ for CD137-mediated innate immunity was examined. IFN-γ is a cytokine that enhances innate immunity against Listeria monocytogenes (LM) infection (Harty & Bevan 1995; Huang et al., 1993). As shown in FIG. 1b, the anti-LM effect of CD137 mAb was completely eliminated in IFN-γ deficient mice. In addition, there was a significant increase in the secretion of IFN-γ from memory T cells upon CD137 mAb treatment in comparison with control mAb. Transfer of purified WT CD3+ T cells into IFN-γ deficient mice was able to restore the effect of CD137 mAb, as shown in FIG. 11b. This data indicates that IFN-γ is needed for the effect of CD137 mAb. The data also implicates that CD137 mAb could induce memory T cells to secrete IFN-γ, therefore contributing to innate immune resistance to LM infection. IFN-γ is not required for the induction of memory T cell proliferation. However, execution of innate immune function of memory T cells requires IFN-γ because the mice with IFN-γ deficiency were not able to eliminate LM infection as shown in FIG. 11. Therefore, proliferation and generation of innate immune functions may be two different processes with distinct requirement for IFN-γ.

To provide direct evidence that CD137 stimulation promotes memory T cells to enhance innate immunity, purified memory OT-1 T cells were transferred into IFN-γ deficient mice. The mice were treated with CD137 mAb or control mAb, and subsequently challenged with LM. As shown in FIG. 11c, upon transfer with memory OT-1 T cells, IFN-γ deficient mice had significantly lower LM titer when treated with CD137 mAb than control mAb, 48 hours after treatment. As OT-1 memory T cells do not cross-react with LM antigen, and significant effect of CD137 mAb is evident within 48 hours, these results support the idea that CD137 mAb is able to directly trigger memory T cells to promote innate immunity against LM infection in an antigen-independent fashion.

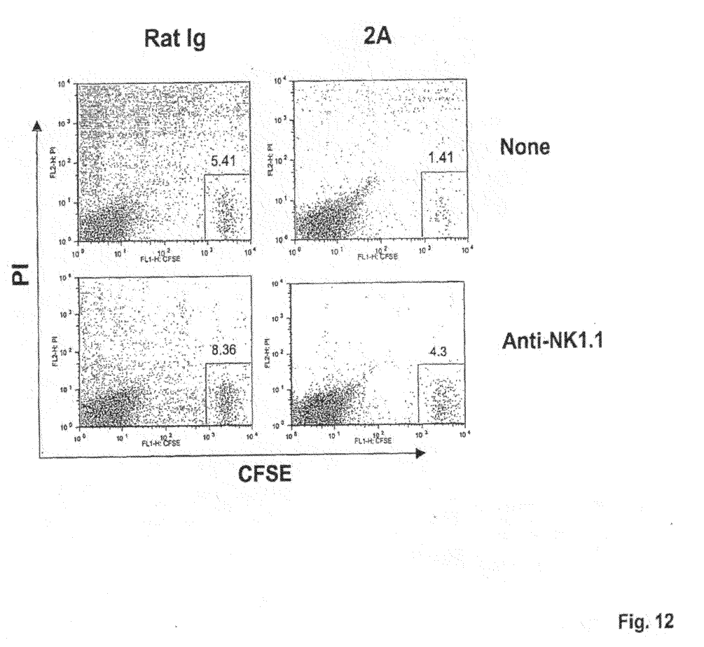

To demonstrate that memory T cell-mediated innate immunity confers a broad spectrum of host defense, whether CD137 mAb treatment is effective in the resistance of tumor challenge was examined. B6 mice were inoculated i.p. with CD137 mAb on day 0 and day 2. At day 7, mice were challenged i.p. with 1×10⁶ CFSE-labeled syngeneic RMA-S lymphoma cells. In comparison with control mAb-treated mice, CD137 mAb-treated mice contained significantly fewer tumor cells 24 hours after challenge (5.12% vs. 0.85% of total peritoneal cells), indicating that CD137 mAb induces a rapid innate immunity against RMA-S. In 48 hours, virtually all RMA-S cells were eliminated, as shown in FIGS. 11d and e. In contrast, similar CD137 mAb treatment did not confer significant resistance to RMA-S tumor challenge in lymphocyte-deficient RAG-1 KO mice, as shown in FIG. 11f. The effect of CD137 mAb appears to be dependent on NK cells because depletion by anti-NK1.1 mAb partially eliminated its antitumor effect, as shown in FIG. 12. Moreover, B6 mice, which had survived from initial RMA-S tumor challenge due to CD137 mAb-induced innate immunity, were highly resistant to challenge by lethal dose RMA-S cells even thirty days later, suggesting the induction of memory T cell immunity. There were no significant changes in numbers of NK, NKT or B cells in the spleens, lymph nodes and livers of wt B6 mice upon CD137 mAb treatment.

The data demonstrates that CD137 mAb does not induce innate immunity against LM infection in the IFN-γ deficient mice, whereas transfer of wild type T cells (FIG. 6b) or memory OT-1 T cells (FIG. 11c) could restore the effect, supporting a direct role of CD137 signal in the stimulation of memory T cells for innate immunity by induction of IFN-γ. Interestingly, depletion of NK/NKT cells by NK1.1 mAb partially inhibited the antitumor effect of CD137 mAb against RMA-S. RMA-S tumor cells are susceptible to NK cell lysis (van den Broek, et al., 1995), and it is possible that secreted IFN-γ from memory T cells may play a role in the activation of NK or NKT cells to induce a resistance to RMA-S tumor. It has been reported that memory CD8+ T cells exhibit characteristics of both T cells and NK cells (Dhanji et al., 2003; McMahon & Raulet 2001), and could potentially mediate innate immunity through secretion of IFN-γ (Berg et al., 2003). The results presented herein support the notion that the effect of CD137 mAb is also mediated through enhancing memory T cell proliferation and cytokine secretion.

Taken together, the data demonstrates that CD137 stimulation alone is sufficient to stimulate growth of memory T cells and acquisition of innate immune function against LM infection and RMA-S tumor growth. Moreover, the results show that CD137 on T cells, upon engagement by agonist mAb, induces Vigorous proliferation of memory but not naïve T cells. Importantly, memory T cells triggered by CD137 signal also acquire effector function for the resistance to LM infection and RMA-S lymphoma challenge. Thus, the CD137 signal is an important factor for growth and function of memory T cells.

The results reported herein were obtained using the following methods and materials.

### Mice

6˜8-week-old C57BL/6 (B6), C3H/HeJ, B6/Thy1.1 and B6/IFN-γ knock out (KO) mice were obtained from the Jackson Laboratory. B6H-2 K^{b}\ This KO strain was used to prove that H-2 Kb is not required for the effect . . . KO, IL15 KO and OT-1 x RAG-1 KO mice were purchased from Taconic Farms. To generate CD137-deficient mice, a 5.1 kb DNA fragment upstream of exon 1 and a 4.8 kb DNA fragment downstream of exon 6 of murine CD137 genomic DNA were PCR amplified from a 129SvJ bacterial artificial chromosome (BAC) library (Invitrogen, Carlsbad, Calif.). The fragments were cloned into a gene-targeting vector, pKOscrambler NTKV-1907, that provides two “scrambled” polylinkers for bidirectional subcloning of mouse genomic fragments as well as insertion sites for selection markers, pKOscrambler NTKV-1907 (Stratagene, La Jolla, Calif.) to generate a targeting plasmid resulting in removing 6 exons from CD137 gene. The targeting fragment containing 5′ arm and 3′arm of CD137, a positive selection marker neomycin (NEO), and a negative selection herpes simplex virus TK (thymidine kinase) genes was transfected into embryonic stem cells from 129Sv mouse strain. Southern blots were used to confirm gene targeting of positive clones. Chimeric mice were produced by injection of targeted embryonic stem cells into blastocysts of B6 hosts. Heterozygous mice were obtained from breeding chimeric mice with B6 mice. Homozygous mice were produced by back-crossing to B6 for more than five generations.

### Antibodies

The following antibodies were purchased from Pharmingen (San Diego, Calif.): CD8-Cy-Chrome™, CD4-Cy-Chrome™, Thy1.2-fluorescein isothiocyanate (FITC), CD44-phycoerythrin (PE), CD62L-FITC, CD122-PE, PD-1-PE, CD137-PE and a FITC bromodeoxyuridine (BrdU) it. SIINFEKL/H-2 Kb-PE tetramer (OT-1 tetramer) was bought from Beckman Coulter, Inc. anti-H-2 Kb mAb (clone AF6.88.5) was bought from ATCC. The generation and purification of CD137 mAb (clone 2A) was described previously (Wilcox et al., 2002a).

### Cell Division Measurement In Vivo

Mice were injected with 100 mg CD137 mAb or Rat IgG (Sigma, St. Louis, Mo.) on day 0 and day 2. On day 3, treated mice were given BrdU (Sigma, St. Louis, Mo.) in drinking water at a concentration of 0.8 mg/ml. On day 7, spleen and liver lymphocytes were prepared as previously described (Dong et al., 2004). All samples were preincubated for 15 min with anti-CD32 and subsequently stained for 30 min at 4° C. with antibodies. After cell-surface staining, intracellular BrdU staining was done using methods known in the art.

### T-Cell Homeostatic Proliferation Assay

Spleens and lymph nodes (LNs) were harvested from OT-1 x RAG1 KO mice and CD8+ T cells were purified using CD8+ T Cell Isolation Kit (Miltenyi Biotec). The donor cells were labeled with CFSE as previous described (Luo et al., 2004). Briefly, cells were suspended in PBS at 2×10⁷/ml and incubated in 5 mM CFSE solution for 15 min at 37° C. Cells were harvested and further incubated in RPMI 1640 medium for 30 min at 37° C. After incubation, donor cells were washed with HBSS twice. 1×10⁶ labeled cells in 0.5 ml HBSS were transferred into B6 hosts that had been irradiated with 600 cGy. Mice were injected intraperitoneally (i.p.) with 100 mg CD137 mAb or control mAb on day 0 or day 7 after adoptive transfer. Spleen cells were harvested and analyzed by flow cytometry six days after antibody injection.

Listeria monocytogenes Challenge Experiments

6 to 8 week-old mice pretreated with CD137 mAb or control Ab on day 7 and −5 were injected i.p. with L. monocytogenes in 400 μl PBS. Survival of the mice was followed daily for two weeks. For determination of bacterial recovery, mice were killed and the livers and spleens were homogenized in PBS. Serial dilutions of homogenates were plated on BHI/streptomycin agar plates and colonies were counted after growth at 37° C. for 24-36 hours.

### Tumor Inoculation In Vivo

RMA-S tumor cells were labeled with CFSE and injected i.p. into mice which were pretreated with either 100 mg/mouse anti-CD137 mAb or Rat IgG on day-7 and day-5 prior to tumor challenge. 24 or 48 hours later, peritoneal cells were washed out with 2×5 ml PBS and counted. The percentage of live RMA-S cells were detected by FACS staining of CFSE-positive cells in total propidium iodide-negative cells.

### Preparation of Memory T Cells In Vivo

The method was described previously with small modifications (Bathe et al., 2001). Briefly, 1×10⁶/ml OT-1 cells were incubated with irradiated CD80/EG7, an EL4 mouse tumor line transfected to express chicken ovalbumin (OVA) and murine CD80 (K. Tamada et al unpublished data) in RPMI medium at ratio 4:1 for 48 hours. Live cells were isolated using LYMPHOLYTE-M (Cedarlane) and incubated in medium containing 20 IU recombinant IL-2 for additional 3 days. Cells were isolated and 1˜2×10⁷ cells were transferred into naïve B6 mice. More than 40 days after transfer, the OT-1 cells were purified and used as memory T cells. In some experiment, CD8+ T cells containing about 20-30% memory OT-1 cells were purified using CD8+ T Cell Isolation Kit (Miltenyi Biotec) and were labeled with CFSE. The labeled cells in HBSS were transferred into H-2 Kb KO mice. CD137 mAb or control mAb were injected on day 0 and day 2. Spleen cells were harvested and gated for CD8 and OT-1 tetramer. Cell division was traced by CFSE dilution analysis.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

Bathe, O. F., Dalyot-Herman, N., and Malek, T. R. (2001). IL-2 during in vitro priming promotes subsequent engraftment and successful adoptive tumor immunotherapy by persistent memory phenotypic CD8+ T cells. J. Immunol. 167, 4511-4517.
Becker, T. C., Wherry, E. J., Boone, D., Murali-Krishna, K., Antia, R., Ma, A., and Ahmed, R. (2002). Interleukin-15 is required for proliferative renewal of virus-specific memory CD8 T cells. J. Exp. Med. 195, 1541-1548.
Berg, R. E., Crossley, E., Murray, S., and Forman, J. (2003). Memory CD8+ T cells provide innate immune protection against Listeria monocytogenes in the absence of cognate antigen. J. Exp. Med. 198, 1583-1593.
Bertram, E. M., Lau, P., and Watts, T. H. (2002). Temporal segregation of CD137 versus CD28-mediated co-stimulation: CD137 ligand influences T cell numbers late in the primary response and regulates the size of the T cell memory response following influenza infection. J. Immunol. 168, 3777-3785.
Bertram, E. M., Dawicki, W., Sedgmen, B., Bramson, J. L., Lynch, D. H., and Watts, T. H. (2004). A switch in costimulation from CD28 to 4-1BB during primary versus secondary CD8 T cell response to influenza in vivo. J. Immunol. 172, 981-988.
Cannons, J. L., Choi, Y., and Watts, T. H. (2000). Role of TNF receptor-associated factor 2 and p38 nitrogen-activated protein kinase activation during 4-1BB-dependent immune response. J. Immunol. 165, 6193 6204.
Chen, L. (2002). Antibody gene therapy: old wine in a new bottle. Nat. Med. 8, 333-334.
Cho, B. K, Rao, V. P., Ge, Q., Eisen, H. N. and Chen, J. (2000). Homeostasis-stimulated proliferation drives naive T cells to differentiate directly into memory T cells. J. Exp. Med. 192, 549-556.
Croft, M. (2003). Co-stimulatory members of the TNFR family: keys to effective T-cell immunity? Nat. Rev. Immunol. 3, 609-620.
Dhanji, S., and Teh, H. S. (2003). IL 2-activated CD8+CD44high cells express both adaptive and innate immune system receptors and demonstrate specificity for syngeneic tumor cells. J. Immunol. 171, 3442-3450.
Dong, H., Zhu, G., Tamada, K, Flies, D. B., van Deursen, J. M., and Chen, L. (2004). B7-H1 determines accumulation and deletion of intrahepatic CD8(+) T lymphocytes. Immunity 20, 327-336.
Dutton, R. W., Bradley, L. M., and Swain, S. L. (1998). T cell memory. Annu. Rev. Immunol. 16, 201-223.
Foell, J., Strahotin, S., O'Neil, S. P., McCausland, M. M., Suwyn, C., Haber, M., Chander, P. N., Bapat, A. S., Yan, X. J., Chiorazzi, N., et al. (2003). CD137 costimulatory T cell receptor engagement reverses acute disease in lupus-prone NZB x NZW F1 mice. J. Clin. Invest. 111, 1505-1518.
Fukushima, A., Yamaguchi, T., Ishida, W., Fukata, K., Mittler, R. S., Yagita, H., and Ueno, H. (2005). Engagement of 4-1BB Inhibits the Development of Experimental Allergic Conjunctivitis in Mice. J. Immunol. 175, 4897-4903.
Futagawa, T., Akiba, H., Kodama, T., Takeda, K, Hosoda, Y., Yagita, H., and Okumura, K. (2002). Expression and function of 4-1BB and 4-1BB ligand on murine dendritic cells. Int. Immunol. 14, 275-286.
Goldrath, A. W., Bogatzki, L. Y. and Bevan, M. J. (2000). Naive T cells transiently acquire a memory-like phenotype during homeostasis-driven proliferation. J. Exp. Med. 192, 557-564.
Goldrath, A. W., Sivakumar, P. V., Glaccum, M., Kennedy, M. K., Bevan, M. J., Benoist, C., Mathis, D., and Butz, E. A. (2002). Cytokine requirements for acute and basal homeostatic proliferation of naive and memory CD8+ T cells. J. Exp. Med. 195, 1515-1522.
Halstead, E. S., Mueller, Y. M., Altman, J. D., and Katsikis, P. D. (2002). In vivo stimulation of CD137 broadens primary antiviral CD8+ T cell responses. Nat. Immunol. 3, 536-541.
Harty, J. T., and Bevan, M. J. (1995). Specific immunity to Listeria monocytogenes in the absence of IFN gamma. Immunity. 3, 109-117.
Hendriks, J., Xiao, Y., Rossen, J. W., van der Sluijs, K. F., Sugamura, K., Ishii, N., and Borst, J. (2005). During Viral Infection of the Respiratory Tract, CD27, 4-1BB, and OX40 Collectively Determine Formation of CD8+ Memory T Cells and Their Capacity for Secondary Expansion. J. Immunol. 175, 1665-1676.
Hoogenboom H R, Winter G. By-passing immunisation. Human antibodies from synthetic repertoires of germiline VH gene segments rearranged in vitro. J Mol Biol 1992; 227: 381-8.
Huang, S., W. Hendriks, A. Althage, S. Hemmi, H. Bluethmann, R. Kamijo, J. Vilcek, R. M. Zinkernagel, and M. Aguet 1993. Immune response in mice that lack the interferon-gamma receptor. Science. 259, 1742-1745.
Jameson, S. C. (2002). Maintaining the norm: T-cell homeostasis. Nat. Rev. Immunol. 2, 547-556.
Kieper, W. C., and Jameson, S. C. (1999). Homeostatic expansion and phenotypic conversion of naive T cells in response to self peptide/MHC ligands. Proc. Natl. Acad. Sci. USA. 96, 13306-13311.
Kieper, W. C., Tan, J. T., Bondi-Boyd, B., Gapin, L., Sprent, J., Ceredig, R, and Surh, C. D. (2002). Overexpression of interleukin (IL)-7 leads to IL-15-independent generation of memory phenotype CD8+ T cells. J. Exp. Med. 195, 1533-1539.
Kim, J., Choi, W. S., La, S., Suh, J. H., Kim, B. S., Cho, H. R., Kwon, B. S., and Kwon, B. (2005). Stimulation with 4-1BB (CD137) inhibits chronic graft-versus-host disease by inducing activation-induced cell death of donor CD4+ T cells. Blood. 105, 2206-2213.
Kwon, B. S., Hurtado, J. C., Lee, Z. H., Kwack, K. B., Seo, S. K., Choi, B. K., Koller, B. H., Wolisi, G., Broxmeyer, H. E., and Vinay, D. S. (2002). Immune responses in 4-1BB (CD137)-deficient mice. J. Immunol. 168, 5483-5490.
Laffly E, Sodoyer R Monoclonal and recombinant antibodies, 30 years after . . . . Hum Antibodies 2005; 14: 33-55.
Luo, L., Chapoval, A. I., Flies, D. B., Zhu, G., Hirano, F., Wang, S., Lau, J. S., Dong, H., Tamada, K, Flies, A. S., Liu, Y., and Chen, L. (2004). B7-H3 enhances tumor immunity in vivo by costimulating rapid clonal expansion of antigen-specific CD8+ cytolytic T cells. J. Immunol. 173, 5445-5450.
McMahon, C. W., and Raulet, D. H. (2001), Expression and function of NK cell receptors in CD8+ T cells. Curr. Opin. Immunol. 13, 465-470.
Miller K D, Weaver-Feldhaus J, Gray S A, Siegel R W, Feldhaus M J. Production, purification, and characterization of human scFv antibodies expressed in Saccharomyces cerevisiae, Pichia pastoris, and Escherichia coli. Protein Expr Purif 2005; 42: 255-67.
Murali-Krishna, K., and Ahmed, R (2000). Cutting edge: naive T cells masquerading as memory cells. J Immunol. 165, 1733-1737.
Nam, K. O., Kang, H., Shin, S. M., Cho, K. H., Kwon, B., Kwon, B. S., Kim, S. J. and Lee, H. W. (2005). Cross-linking of 4-1BB activates TCR-signaling pathways in CD8+ T lymphocytes. J. Immunol. 174, 1898-1905
Okazaki, T., Iwai, Y., and Honjo, T. (2002). New regulatory co-receptors: inducible co-stimulator and PD-1. Curr. Opin. Immunol. 14, 779-782.
Saoulli, K., Lee, S. Y., Cannons, J. L., Yeh, W. C., Santana, A., Goldstein, M. D., Bangia, N., DeBenedette, M. A., Mak, T. W., Choi, Y., and Watts, T. H. (1998). CD28-independent, TRAF2-dependent costimulation of resting T cells by 4-1 BB ligand. J. Exp. Med. 187, 1849-1862.
Schluns, K. S., and Lefrancois, L. (2003). Cytokine control of memory T-cell development and survival. Nat. Rev. Immunol. 3, 269-279.
Seddon, B., Tomlinson, P., and Zamoyska, R. (2003). Interleukin 7 and T cell receptor signals regulate homeostasis of CD4 memory cells. Nat. Immunol. 4, 680-686.
Seo, S. K., Choi, J. H., Kim, Y. H., Kang, W. J., Park, H. Y., Suh, J. H., Choi, B. K., Vinay, D. S., and Kwon, B. S. (2004). 4-1BB-mediated immunotherapy of rheumatoid arthritis. Nat. Med. 10, 1088-1094.
Sun, Y., Chen, H. M., Subudhi, S. K., Chen, J., Koka, R., Chen, L., and Fu, Y. X. (2002). Costimulatory molecule-targeted antibody therapy of a spontaneous autoimmune disease. Nat. Med. 8, 1405-1413.
Tan, J. T., Whitmire, J. K., Ahmed, R., Pearson, T. C. and Larsen, C. P. (1999). CD137 ligand, a member of the TNF family, is important for the generation of antiviral CD8 T cell responses. J. Immunol. 163, 4859-4868.
Tough, D. F., Sun, S., and Sprent, J. (1997). T cell stimulation in vivo by lipopolysaccharide (LPS). J. Exp. Med. 185, 2089-2094.
Van den Broek, M. F., Kagi, D., Zinkernagel, R. M. and Hengartner, H. (1995). Perforin dependence of natural killer cell-mediated tumor control in vivo. Eur. J. Immunol. 25, 3514-3516
Vaughan T J, Williams A J, Pritchard K, Osbourn J K, Pope A R, Earnshaw J C, McCafferty J, Hodits R A, Wilton J, Johnson K S. Human antibodies with sub-nanomolar affinities isolated from a large non-immunized phage display library. Nat Biotechnol 1996; 14: 309-14.
Watts, T. H. (2005). TNF/TNFR family members in costimulation of T cell responses. Annu. Rev. Immunol. 23, 23-68.
Wilcox, R. A., Flies, D. B., Zhu, G., Johnson, A. J., Tamada, K., Chapoval, A. I., Strome, S. E., Pease, L. R., and Chen, L. (2002a). Provision of antigen and CD137 signaling breaks immunological ignorance, promoting regression of poorly immunogenic tumors. J. Clin. Invest. 109, 651-659.
Wilcox, R. A., Chapoval, A. I., Gorski, K. S., Otsuji, M., Shin, T., Flies, D. B., Tamada, K, Mittler, R. S., Tsuchiya, H., Pardoll, D. M., and Chen, L. (2002b). Cutting edge: Expression of functional CD137 receptor by dendritic cells. J. Immunol. 168, 4262-4267.
Wilcox, R. A., Tamada, K., Strome, S. E., and Chen, L. (2002c). Signaling through NK cell-associated CD137 promotes both helper function for CD8+ cytolytic T cells and responsiveness to IL-2 but not cytolytic activity. J. Immunol. 169, 4230-4236.
Wilcox, R. A., Tamada, K., Flies, D. B., Zhu, G., Chapoval, A. I., Blazar, B. R., Kast, W. M., and Chen, L. (2004). Ligation of CD137 receptor prevents and reverses established anergy of CD8+ cytolytic T lymphocytes in vivo. Blood 103, 177-184.
Winter G, Griffiths A D, Hawkins R E, Hoogenboom HR. Making antibodies by phage display technology. Annu Rev Immunol 1994; 12: 433-55.
Zhang, X., Sun, S., Hwang, I., Tough, D. P., and Sprent, J. (1998). Potent and selective stimulation of memory-phenotype CD8+ T cell in vivo by IL-15. Immunity 8, 591-599.
Zhu, Y., and Chen, L. (2003). Cancer therapeutic monoclonal antibodies targeting lymphocyte co-stimulatory pathways. Curr. Opin. Invest. Drugs 4, 691-695.

1. A method of increasing innate immune function in a subject identified as in need thereof, the method comprising
(a) contacting a memory T cell of the subject with an agent that specifically binds CD137; and
(b) inducing memory T cell proliferation in the subject, thereby increasing innate immunity.

2. The method of claim 1, wherein the method prevents a neoplasia or pathogen infection in a subject at risk thereof.

3. A method of increasing memory T cell proliferation, the method comprising
(a) contacting a memory T cell expressing CD137 with an agent that activates CD137; and
(b) inducing memory T cell proliferation.

4. The method of claim 1, wherein the method induces cytokine secretion or cytolytic activity in tumor cells.

5. The method of claim 1, wherein memory T cell proliferation is independent of T cell receptor signalling.

6. A method of treating or preventing a pathogen infection in a subject in need thereof, the method comprising:
(a) administering to the subject an agent that specifically binds CD137 on a memory T cell; and
(b) inducing an innate immune response in the subject, thereby treating or preventing a pathogen infection.

7. The method of claim 6, wherein the pathogen infection is bacterial, viral, or fungal.

8. The method of claim 4, wherein the bacterial infection is an infection with selected from the group consisting of Aerobacter, Aeromonas, Acinetobacter, Actinomyces israelii, Agrobacterium, Bacillus, Bacillus antracis, Bacteroides, Bartonella, Bordetella, Bortella, Borrelia, Brucella, Burkholderia, Calymmatobacterium, Campylobacter, Citrobacter, Clostridium, Clostridium perfringers, Clostridium tetani, Cornyebacterium, corynebacterium diphtheriae, corynebacterium sp., Enterobacter, Enterobacter aerogenes, Enterococcus, Erysipelothrix rhusiopathiae, Escherichia, Francisella, Fusobacterium nucleatum, Gardnerella, Haemophilus, Hafnia, Helicobacter, Klebsiella, Klebsiella pneumoniae, Lactobacillus, Legionella, Leptospira, Listeria (e.g., Listeria monocytogenes), Morganella, Moraxella, Mycobacterium, Neisseria, Pasteurella, Pasturella multocida, Proteus, Providencia, Pseudomonas, Rickettsia, Salmonella, Serratia, Shigella, Staphylococcus, Stentorophomonas, Streptococcus, Streptobacillus moniliformis, Treponema, Treponema pallidium, Treponema pertenue, Xanthomonas, Vibrio, and Yersinia.

9. The method of claim 8, wherein the bacteria is Listeria monocytogenes.

10. The method of claim 8, wherein the viral infection is an infection with a virus selected from the group consisting of Retroviridae, HIV-1, Picornaviridae, Calciviridae, Flaviridae, Coronoviridae, Filoviridae, Paramyxoviridae, Orthomyxoviridae, Bungaviridae, Arena viridae, Birnaviridae; Hepadnaviridae, Parvovirida, Papovaviridae, Adenoviridae, Herpesviridae, Poxyiridae and Iridoviridae.

11. The method of claim 11, wherein the viral infection is an Human immunodeficiency virus infection.

12. A method of treating or preventing a neoplasia in a subject in need thereof, the method comprising:
(a) administering to the subject an agent that specifically binds CD137 on a memory T cell; and
(b) inducing an innate immune response in the subject, thereby treating or preventing a neoplasia.

13. The method of claim 13, wherein the neoplasia is selected from the group consisting of lymphoma, melanoma, breast, ovarian, prostate, colon, and brain cancers.

14-26. (canceled)

27. A method for increasing homeostatic proliferation in a subject identified as in need thereof, the method comprising
(a) contacting a memory T cell expressing CD137 with an agent that activates CD137; and
(b) inducing memory T cell proliferation.

28. The method of claim 27, wherein the subject is undergoing chemotherapy.

29. The method of claim 27, wherein the subject is diagnosed with a lymphopenia.

30-31. (canceled)

32. A method for identifying an agent that modulates innate immunity, the method comprising the steps of:
(a) providing a cell expressing a CD137 nucleic acid molecule;
(b) contacting the cell with a candidate compound; and
(c) comparing CD137 nucleic acid molecule expression in the contacted cell with a reference level of expression, wherein an alteration in CD137 nucleic acid molecule expression identifies the candidate compound as a candidate compound that modulates innate immunity.

33-43. (canceled)

44-47. (canceled)

48. A method of screening for a compound that modulates an immune response, the method comprising, exposing the mouse of claim 43, or a cell derived therefrom, to a compound, and determining the level of immune response in the mouse, wherein an increase in the immune response as compared to an untreated mouse indicates that the compound enhances an immune response.

49. A method of producing the mouse of claim 43, the method comprising:
(a) generating a targeting plasmid comprising a CD137 gene comprising a mutation;
(b) contacting an embryonic stem cell of a wild type mouse with the targeting plasmid;
(c) injecting the targeted embryonic stem cell into a blastocyst of a host mouse to produce a zygote;
(d) transplanting the zygote into a host mouse;
(e) obtaining a founder mouse carrying the knockout; and
(f) breeding the founder mouse to obtain a mouse that lacks detectable levels of CD137.

50. An isolated antibody that specifically binds human CD137.

51. The antibody of claim 50, wherein the antibody is a monoclonal antibody that acts as a CD137 agonist.


SEQUENCE LISTING (4 sequences)

SEQ ID NO: 1 - DNA, 2350 bp, Mus musculus
  modified_base (1253)..(1255): a, c, g, t, unknown or other
atgtccatgaactgctgagtggataaacagcacgggatatctctgtctaaaggaatattactacaccaggaaaaggacacattcgacaacaggaaaggagcctgtcacagaaaaccacagtgtcctgtgcatgtgacatttcgccatgggaaacaactgttacaacgtggtggtcattgtgctgctgctagtgggctgtgagaaggtgggagccgtgcagaactcctgtgataactgtcagcctggtactttctgcagaaaatacaatccagtctgcaagagctgccctccaagtaccttctccagcataggtggacagccgaactgtaacatctgcagagtgtgtgcaggctatttcaggttcaagaagttttgctcctctacccacaacgcggagtgtgagtgcattgaaggattccattgcttggggccacagtgcaccagatgtgaaaaggactgcaggcctggccaggagctaacgaagcagggttgcaaaacctgtagcttgggaacatttaatgaccagaacggtactggcgtctgtcgaccctggacgaactgctctctagacggaaggtctgtgcttaagaccgggaccacggagaaggacgtggtgtgtggaccccctgtggtgagcttctctcccagtaccaccatttctgtgactccagagggaggaccaggagggcactccttgcaggtccttaccttgttcctggcgctgacatcggctttgctgctggccctgatcttcattactctcctgttctctgtgctcaaatggatcaggaaaaaattcccccacatattcaagcaaccatttaagaagaccactggagcagctcaagaggaagatgcttgtagctgccgatgtccacaggaagaagaaggaggaggaggaggctatgagctgtgatgtactatcctaggagatgtgtgggccgaaaccgagaagcactaggaccccaccatcctgtggaacagcacaagcaaccccaccaccctgttcttacacatcatcctagatgatgtgtgggcgcgcacctcatccaagtctcttctaacgctaacatatttgtctttaccttttttaaatctttttttaaatttaaattttatgtgtgtgagtgttttgcctgcctgtatgcacacgtgtgtgtgtgtgtgtgtgtgacactcctgatgcctgaggaggtcagaagagaaagggttggttccataagaactggagttatggatggctgtgagccggnnngataggtcgggacggagacctgtcttcttattttaacgtgactgtataataaaaaaaaaatgatatttcgggaattgtagagattctcctgacacccttctagttaatgatctaagaggaattgttgatacgtagtatactgtatatgtgtatgtatatgtatatgtatatataagactcttttactgtcaaagtcaacctagagtgtctggttaccaggtcaattttattggacattttacgtcacacacacacacacacacacacacacacgtttatactacgtactgttatcggtattctacgtcatataatgggatagggtaaaaggaaaccaaagagtgagtgatattattgtggaggtgacagactaccccttctgggtacgtagggacagacctccttcggactgtctaaaactccccttagaagtctcgtcaagttcccggacgaagaggacagaggagacacagtccgaaaagttatttttccggcaaatcctttccctgtttcgtgacactccaccccttgtggacacttgagtgtcatccttgcgccggaaggtcaggtggtacccgtctgtaggggcggggagacagagccgcgggggagctacgagaatcgactcacagggcgccccgggcttcgcaaatgaaacttttttaatctcacaagtttcgtccgggctcggcggacctatggcgtcgatccttattaccttatcctggcgccaagataaaacaaccaaaagccttgactccggtactaattctccctgccggcccccgtaagcataacgcggcgatctccactttaagaacctggccgcgttctgcctggtctcgctttcgtaaacggttcttacaaaagtaattagttcttgctttcagcctccaagcttctgctagtctatggcagcatcaaggctggtatttgctacggctgaccgctacgccgccgcaataagggtactgggcggcccgtcgaaggccctttggtttcagaaacccaaggcccccctcataccaacgtttcgactttgattcttgccggtacgtggtggtgggtgccttagctctttctcgatagttagac

SEQ ID NO: 2 - protein, 256 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 3 - DNA, 1935 bp, Homo sapiens
agaccaaggagtggaaagttctccggcagccctgagatctcaagagtgacatttgtgagaccagctaatttgattaaaattctcttggaatcagctttgctagtatcatacctgtgccagatttcatcatgggaaacagctgttacaacatagtagccactctgttgctggtcctcaactttgagaggacaagatcattgcaggatccttgtagtaactgcccagctggtacattctgtgataataacaggaatcagatttgcagtccctgtcctccaaatagtttctccagcgcaggtggacaaaggacctgtgacatatgcaggcagtgtaaaggtgttttcaggaccaggaaggagtgttcctccaccagcaatgcagagtgtgactgcactccagggtttcactgcctgggggcaggatgcagcatgtgtgaacaggattgtaaacaaggtcaagaactgacaaaaaaaggttgtaaagactgttgctttgggacatttaacgatcagaaacgtggcatctgtcgaccctggacaaactgttctttggatggaaagtctgtgcttgtgaatgggacgaaggagagggacgtggtctgtggaccatctccagccgacctctctccgggagcatcctctgtgaccccgcctgcccctgcgagagagccaggacactctccgcagatcatctccttctttcttgcgctgacgtcgactgcgttgctcttcctgctgttcttcctcacgctccgtttctctgttgttaaacggggcagaaagaaactcctgtatatattcaaacaaccatttatgagaccagtacaaactactcaagaggaagatggctgtagctgccgatttccagaagaagaagaaggaggatgtgaactgtgaaatggaagtcaatagggctgttgggactttcttgaaaagaagcaaggaaatatgagtcatccgctatcacagctttcaaaagcaagaacaccatcctacataatacccaggattcccccaacacacgttcttttctaaatgccaatgagttggcctttaaaaatgcaccactttttttttttttttgacagggtctcactctgtcacccaggctggagtgcagtggcaccaccatggctctctgcagccttgacctctgggagctcaagtgatcctcctgcctcagtctcctgagtagctggaactacaaggaagggccaccacacctgactaacttttttgttttttgtttggtaaagatggcatttcaccatgttgtacaggctggtctcaaactcctaggttcactttggcctcccaaagtgctgggattacagacatgaactgccaggcccggccaaaataatgcaccacttttaacagaacagacagatgaggacagagctggtgataaaaaaaaaaaaaaaaaagcattttctagataccacttaacaggtttgagctagtttttttgaaatccaaagaaaattatagtttaaattcaattacatagtccagtggtccaactataattataatcaaaatcaatgcaggtttgttttttggtgctaatatgacatatgacaataagccacgaggtgcagtaagtacccgactaaagtttccgtgggttctgtcatgtaacacgacatgctccaccgtcaggggggagtatgagcagagtgcctgagtttagggtcaaggacaaaaaacctcaggcctggaggaagttttggaaagagttcaagtgtctgtatatcctatggtcttctccatcctcacaccttctgcctttgtcctgctcccttttaagccaggttacattctaaaaattcttaacttttaacataatattttataccaaagccaataaatgaactgcatatgaaaaaaaaaaaaaaaaaaaaaaaaaaaaaaaaaaaaaaaaaaaaaaaaaaaa

SEQ ID NO: 4 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL